(19) United States
(12) Patent Application Publication
(10) Pub. No.: US 2020/0352206 A1
(43) Pub. Date: Nov. 12, 2020

(54) COMPOSITION AND METHOD FOR MUSHROOM MYCELIAL COMPOUND AND BOTANICAL MIXTURE

(71) Applicant: Christine Wagner-Salvini, Sebastian, FL (US)

(72) Inventor: Christine Wagner-Salvini, Sebastian, FL (US)

(21) Appl. No.: 16/869,839

(22) Filed: May 8, 2020

Related U.S. Application Data
(60) Provisional application No. 62/886,382.
(60) Provisional application No. 62/846,626, filed on May 11, 2019.
(60) Provisional application No. 62/845,032, filed on May 8, 2019.

(51) Int. Cl. A23L 31/00; A61K 36/07; A61K 36/062; A61K 36/33; A61K 31/05; A23L 5/20; A23L 33/105

(52) U.S. Cl. CPC A23L 31/00; A61K 36/07; A61K 36/062; A23L 33/105; A61K 31/05; A23L 5/21; A61K 36/33

(57) ABSTRACT

Described is a multi-mushroom mycelial compound and botanical mixture, formulated as a solid or liquid composition, and methods of using the composition having nutritional and mood enhancing properties. The mushroom composition can include cactus or cactus extract, and can be formulated as a solid or liquid form for ingestion by a patient in need of the benefit provided by the composition.

### CROSS-REFERENCE TO RELATED APPLICATIONS

This application claims the benefit of U.S. Provisional Patent Application Ser. No. 62/845,032, filed May 8, 2019. This application also claims the benefit of U.S. Provisional Patent Application Ser. No. 62/846,626, filed May 11, 2019. This application also claims the benefit of U.S. Provisional Patent Application Ser. No. 62/886,382, filed Jun. 19, 2019. The above US Provisional Patent Applications are hereby incorporated by reference, in their entirety.

### BACKGROUND OF THE INVENTION

A medical diagnosis of a serious or life-threatening disease or condition can have a huge impact on most patients, families, and caregivers. Feelings of depression, anxiety, and fear are common and are normal responses to a life-changing experience. Many things can cause these feelings. Changes in body image can affect self-esteem and confidence. Family and work roles may be altered. People might feel grief at these losses and changes. Physical symptoms such as pain, nausea, or extreme fatigue also seem more likely to cause emotional distress. People might also fear death, suffering, pain, or all the unknown things that lie ahead. Cannabis has proven to aid with many symptoms and conditions but may not help entirely.

Neurodegenerative disease is an umbrella term for a range of conditions which primarily affect the neurons in the human brain. Neurons are the building blocks of the nervous system which includes the brain and spinal cord. Neurons normally do not reproduce or replace themselves, so when they become damaged or die, they cannot be replaced by the body causing problematic symptoms. Symptoms vary in degree, changing from one moment to the next like a broken electrical wire sending mixed signals. Neurological disorders can affect an entire neurological pathway or a single neuron. A small disturbance to a neuron's structural pathway can result in dysfunction. As a result, neurological disorders can result from a number of causes, including: lifestyle-related causes, infections, genetics, nutrition-related causes, environmental influences, physical injuries.

Physicians face difficulties when diagnosing neurodegenerative disease. The diagnostic tool commonly used to assess cognitive impairment in neurodegenerative diseases is based on established clinical criteria. However, the differential diagnosis between disorders can be difficult, especially in early phases or atypical variants. Upon a medical encounter, a patient may present multiple complaints and chronic conditions warranting the prescription of numerous pharmaceuticals. As a result, a physician may vary and/or increase a patient's use of prescription pharmaceuticals which can cause a network of side effects, reactions, and further difficult to diagnose circumstances. The longer a patient is symptomatic, the higher the risk that they will develop additional complications.

While neurological disorders involve damage to and degeneration of the nervous system, sometimes that damage can alter the communication between neurons. When it does, those changes can manifest themselves in problems with behavior, body control, memory, and mood. It is widely believed in the scientific community that problematic communication between neurons in our brains underpins psychiatric disorders.

In the last decade, multiple investigative groups have identified structural changes of various neuroanatomic structures in patients with idiopathic major depression and bipolar disorders. Using high-resolution MRI of the brain and functional neuroimaging studies (i.e., PET, SPECT), researchers have described decreases in the volume of hippocampal formation, amygdala, entorhinal cortex, various frontal lobe structures, and basal ganglia, in addition to abnormal cerebral blood flow and metabolic activity in these structures as well as in thalamic nuclei. Similar structural and functional changes have been identified in patients with depression associated with a variety of neurologic disorders (i.e., stroke, Parkinson's disease, epilepsy, Alzheimer's dementia). In addition, recent data have shown that depression is a risk factor for the development of several neurologic disorders, including epilepsy, stroke, and Parkinson's disease and bears a negative impact on the course and outcome of most neurologic disorders.

Neuronal communication has been implicated in depression, behavioral problems, posttraumatic stress disorder, attention deficit hyperactivity disorder, addictive behaviors, substance abuse, and schizophrenia. These disorders can affect social interactions, mood, concentration, memory, and body control. Psychiatrists are often referred due to their medical training and experience in addressing both the mental and physical contributors to mental illness and their relation to neurodegenerative disease.

Nearly 1 in 5 people will experience a major depressive episode at some point in their lives. PTSD, OCD Fibromyalgia, neuropathic pain, chronic migraines, substance abuse, Bipolar disorder, postpartum syndrome, PMS physical health, psychosocial adversity in childhood, and ongoing or recent psychosocial stress may impact multiple neurobiological systems relevant to major depressive disorder. Similar prescribing methods exist within psychiatry and neurology which often include sedatives, narcotics, Ketamine infusions, therapy, and various types of psychotherapy.

This disparate group of disorders includes pain experienced by both the body and mind: anxiety and depression, complex regional pain syndrome, opioid-induced hyperalgesia, phantom limb pain, fibromyalgia, and PTSD. In trained hands, Ketamine infusions can be beneficial, often providing the first relief for patients frustrated by years of seeking help. However, these treatments come with serious risks.

Cannabis has been shown to provide substantial benefits for medical and recreational uses. Cannabis is used by a wide cross-section of society to treat, conditions and symptoms from Cancer Aids Arthritis IBS and many of the conditions previously mentioned but may not help entirely. By combining Cannabis and the healing power of mushrooms we can create a full spectrum of benefits through methods of cultivation and or symbiotic purpose and relation of combined ingredients. Cannabis has been used in medicine for thousands of years prior to achieving its current illicit substance status. Cannabinoids, the active components of Cannabis sativa, mimic the effects of the endogenous cannabinoids (endocannabinoids), activating specific cannabinoid receptors, particularly CB1 found predominantly in the central nervous system and CB2 found predominantly in cells involved with immune function. Delta-9-tetrahydrocannabinol, the main bioactive cannabinoid in the plant, has been available as a prescription medication approved for treatment of cancer chemotherapy-induced nausea and vomiting and anorexia associated with the AIDS wasting syndrome. Cannabinoids may be of benefit in the treatment of cancer-related pain, possibly synergistic with opioid analgesics. Cannabinoids have been shown to be of benefit in the treatment of HIV-related peripheral neuropathy, suggesting that they may be worthy of study in patients with other neuropathic symptoms. Cannabinoids have a favorable drug safety profile, but their medical use is predominantly limited by their psychoactive effects and their limited bioavailability

Investigations into the neurobiology of major depressive disorders have traditionally focused on the monoamine neurotransmitters serotonin and norepinephrine. The monoamine hypothesis initially posited that depressed individuals are likely to have low levels of these neuro-transmitters because various antidepressant drugs acutely increase their levels. However, even though monoaminergic antidepressants are generally used for first-line treatment, they do not exert their clinical benefit immediately and for some people they do not provide any benefit at all; carry side effects and or interactions with medications posing a web of difficulty for physicians when diagnosing patients.

Many forms of chronic disease such as cancer and cardiovascular disfunction can, in part, be attributed to diet and arising from the awareness of the relationship between diet and disease has evolved the concept of ‘functional foods’. The US Academy of Science has defined functional foods as those that ‘encompass potentially health products’ including ‘any modified food or food ingredient that may provide a health benefit beyond the traditional nutrients it contains’. ‘Foods as medicine’ underpins the paradigm of functional foods. Functional foods cannot claim to cure diseases but, increasingly, evidence is being produced that supports the role of some functional foods in disease prevention. The concept of foods as medicine does not fit easily within the current expertise in either pharmaceutical or food companies and the full creative development of functional foods may well require new alliances between these companies with respect to regulatory issues. Fleshy mushrooms (members of the class Basidiomycetes) have long been valued as highly tasty and nutritional foods by many societies throughout the world. To the ancient Romans they were ‘the foods of the Gods’, to the early Egyptians they were ‘a gift from the God Osiris’ while, more appropriately, the Chinese considered them ‘the elixir of life’. Several ancient societies dating back as far as the Palaeolithic period recognised the psychoactive, hallucinogenic properties of some mushrooms, especially Amanita muscaria and Psilocybe spp., and involved them in ancient religious beliefs and practices.

Many cultures worldwide, but especially in the Orient, recognized that extracts from certain mushrooms could have profound health promoting benefits and, consequently, became essential components in many traditional Chinese medicines. The practice of using medicinal mushrooms in Chinese traditional medicine dates back into antiquity and has been recorded in ancient Chinese manuscripts. Increased scientific and medical research in recent years and published in peer reviewed journals, especially in Japan, Korea, and China, and more recently in the US, is increasingly confirming the medicinal efficacy and identifying the bioactive molecules. Recent advances in chemical technology have allowed the isolation and purification of some of the relevant compounds especially polysaccharides which possess strong immunomodulation and anti-cancer activities. The bioactive polysaccharides isolated from mushroom fruit-bodies, submerged cultured mycelial biomass or liquid culture broths are either water-soluble β-D-glucans, β-D-glucans with heterosaccharide chains of xylose, mannose, galactose or uronic acid, or β-D-glucan-protein complexes i.e. proteoglycans (Mizuno 1999).

While many are orally bioavailable, others are mainly effective only by intraperitoneal injection. The main medically important polysaccharides that have undergone extensive anticancer clinical trials include lentinan (Lentinula edodes), schizophyllan (Schizophyllum commune), PSK (polysaccharide-K, commercially sold as Krestin) and PSP (polysaccharopeptide) (Trametes versicolor), and Grifron-D (Grifola frondosa) All of these compounds are currently produced by Asian pharmaceutical companies. These medicinal polysaccharides are primarily modifiers of biological response where these polymers interact with the immune system to up-regulate or down-regulate specific aspects of the response of the host and this may result in various therapeutic effects. Their ability to enhance or suppress immune responses can depend on a number of factors including dosage, route of administration, timing and frequency of administration, mechanism of action or the site of activity. Several of these compounds have been shown to potentiate the host's innate (non-specific) and acquired (specific) immune responses and to activate many kinds of immune cells that are important for the maintenance of homeostasis, e.g. host cells (such as cytotoxic macrophages, monocytes, neutrophils, natural killer cells, dendritic cells) and chemical messengers (cytokines such as interleukins, interferon, colony stimulating factors) that trigger complement and acute phase responses. They can also be considered as multi-cytokine inducers capable of modulating gene expression of various immunomodulatory cytokines via specific cell membrane receptors. Lymphocytes governing antibody production (β-cells) and cell-mediated cytotoxicity (T-cells) are also stimulated. Anti-cancer properties There have been extensive in vivo studies demonstrating the anti-cancer activity of the extracted, purified glucan polysaccharides and polysaccharide peptides in animal models (Wasser & Weis 1999). These studies strongly suggest an immunomodulating mode of action. However, in in vitro studies on various cancer cell lines, there is evidence for direct cytotoxic effects on the cancer cells for some, but not all, of the polysaccharides. Many of the proprietary mushroom polysaccharides have proceeded through Phase I, II and III clinical trials mainly in Japan and China while some are now occurring in the US. In almost all cases the polysaccharides are used as adjuvant treatments with conventional chemotherapy/radiotherapy with many forms of cancer.

There are at least 270 species of mushrooms that are known to possess various therapeutic properties and the term ‘medicinal mushroom’ is now increasingly gaining worldwide recognition. Edible mushrooms which demonstrate medicinal or functional properties include species of Lentinula, Hericium, Grifola, Flammulina, Pleurotus and Tremella while others known only for their medicinal properties, viz. Ganoderma lucidum and Trametes (Coriolus) versicolor are decidedly non-edible because of their coarse texture and bitter taste. Historically, most medicinal mushroom species were relatively scarce and were collected from the forests where they grew on dead or living trees and forest litter. They are predominantly lignocellulose degraders. For medicinal purposes, they were almost always prepared either as hot water extracts, concentrates or in powdered form. Nowadays almost all of the important medicinal mushrooms have been subjected to large-scale artificial cultivation by solid substrate or low moisture fermentation, thus removing the historical scarcity factor and allowing large commercial operations to develop. Mushroom cultivation is the only large-scale biotechnological process that creatively utilizes lignocellulosics.

Mushrooms are an important source of ingredients for modern medicine and holistic remedies. The phytotherapeutic efficiency of terpenes from mushrooms has been previously proven. For example, mushrooms are a rich source of lanostane type triterpenoids which have potential uses in cancer disease treatments. The demand to isolate the bioactive compounds from mushroom species has been increasing due to their medicinal importance as antibacterial, anticancer, anticandidal, anticholinesterase, anti-compliment, antileishmanial, anti-inflammatory, anti-invasive, antimalarial, antioxidant, antitubercular, antitumor, antiviral, and cytotoxicas.

Modern science recognizes the importance of microbial function in ecosystems. Mycelium is a foundation of nature's land-based food webs. Understanding the role of mushrooms and their mycelia in ecosystems and DNA linkage empowers their use in naturopathic medicine in ways that bolster conventional medical practices.

The arising awareness about functional food has created a rapidly emerging scientific revolution in medicinal mushrooms. Many fungi are superficially plant like organisms. On a microscopic level, plants, and fungi both have cell walls, a feature that metazoan (animal) cells lack. The study of cladistics, results in a phylogeny tree where fungi are more closely related to animals than to plants.

These disorders can affect social interactions, mood, concentration, memory, and body control. With medical training, psychiatrists are often referred being skilled in addressing both the mental and physical contributors to mental illness and relation to neurodegenerative disease. Nearly 1 in 5 people will experience a major depressive episode at some point in their lives. PTSD, OCD, Fibromyalgia, neuropathic pain, Chronic Migraines, Substance abuse, Bipolar, Postpartum syndrome, PMS physical health, psychosocial adversity in childhood, and ongoing or recent psychosocial stress may impact multiple neurobiological systems relevant to major depressive disorder. Similar prescribing methods exist within psychiatry and neurology, which often can be sedatives, narcotics, Ketamine infusions, therapy, and various types of psychotherapy. This disparate group of disorders includes pain experienced by both the body and mind: anxiety and depression, complex regional pain syndrome, opioid-induced hyperalgesia, phantom limb pain, fibromyalgia, and PTSD. In trained hands, Ketamine infusions can benefit, often providing the first relief for patients frustrated by years of seeking help. These treatments come with serious risks as well.

Mushrooms offer tremendous applications as they can be used as food and medicines besides their key ecological roles. They represent as one of the world's greatest untapped resources of nutrition and palatable food of the future. Mushrooms have been found effective against cancer, cholesterol reduction, stress, insomnia, asthma, allergies, and diabetes. Due to high amount of proteins, they can be used to bridge the protein malnutrition gap. Mushrooms as functional foods are used as nutrient supplements to enhance immunity in the form of tablets. Due to low starch content and low cholesterol, they suit diabetic and heart patients. One third of the iron in the mushrooms is in available form. Their polysaccharide content is used as anticancer drug. Even, they have been used to combat HIV effectively. Biologically active compounds from the mushrooms possess antifungal, antibacterial, antioxidant and antiviral properties, and have been used as insecticides and nematicides as well. Thus keeping in view the tremendous applications of mushrooms, the present study reviews different aspects of mushrooms towards human health benefits such as food, medicine, minerals, drugs.

For remedy Mushrooms Cannabis and holistic medicine are important source of drugs, nausea, glaucoma, Crohns, Inflammation, immunological support, epilepsy, Leprosy, PTSD, pain, and disorders and symptoms related to Cancer and or Neurdegenerative disorders. Any taxonomic category of fungi or Cannabis strain alone or combined can offer proven Phytotherapeutic efficiency of terpenes. Lanostane type triterpenoids having potentials of rich sources for cancer and disease treatments Psilocybin classifications should not be excluded, is a hallucinogen that works by activating serotonin receptors, most often in the prefrontal cortex. This part of the brain affects mood, cognition, and perception Therefore, the demand of isolation of bioactive compounds from both Mushrooms and Cannabis have been increasing due to their medicinal importance reviewed isolation and biological activities such as antibacterial, anticancer, anticandidal, anticholinesterase, anti-compliment, antileishmanial, anti-inflammatory, anti-invasive, antimalarial, antioxidant, antitubercular, antitumor, antiviral, and cytotoxicas. Modern science now recognizes the importance of understanding the role of mushroom mycelia in ecosystems; DNA linkage empowers their use in naturopathic medicine in ways that bolster conventional medical practices.

When symbiotic relations are respected and studied within cultivation, products of unparalleled property can establish. Mycelium forms a vegetative part of a fungus consisting of a network of branched, tubular filaments referred to as hyphal structures. The unique mycelial structures play a pivotal role in nutrient absorption by releasing enzymes into the substrate, including enzymes for breaking down complex organic polymers into simpler compounds which are absorbed through hyphal wall.

Mycorrhizae are symbiotic relationships that form between fungi and plants. The fungi colonize the root system of a host plant, providing increased water and nutrient absorption capabilities while the plant provides the fungus with beneficial botanicals formed from photosynthesis. Mycelia mushrooms develop beneficial microbial medicinal properties boasting robust terpenoid profiles through organic/natural environment cultivation, enhanced substrates/mediums in a controlled/natural environment.

Similarly, cacti have a wide range of health benefits. These include the ability to aid in weight loss, cancer prevention, improving skin health, and protecting heart health. Cacti also help regulate and improve digestion, boost the immune system, optimize metabolic activity, build strong bones, cure insomnia, and reduce inflammation throughout the body.

Cactus (Opuntia ficus-indica) has been used in traditional folk medicine in many countries because of its role in treating a number of diseases and conditions including diabetes, hypertension, hypercholesterolemic, rheumatic pain, gastric mucosa diseases and asthma. The cactus, fruits, and cladodes are the focus of many studies because they contain bioactive (phytochemicals) compounds, well known for their health-related properties. There exists a positive correlation between a diet rich in prickly pear cactus and a reduced risk of diseases associated with oxidative stress such as diabetes, cancer, cardiovascular, and neurodegenerative diseases. The Opuntia ficus-indica exhibits diverse pharmacological actions through its antioxidant activity: protects cells against oxidative damage, acts as radical scavengers, reduces lipid peroxidation, and increases GSH levels. There are no reported cases of O. ficus-indica having any adverse/toxic effects on humans. More studies on Opuntia ficus-indica could help better understand its pharmacological mechanism of action to provide clear scientific evidence to explain its traditional uses, and to identify its therapeutic potential in other diseases.

The medicinal use of mushrooms has deep set roots in traditional Eastern medicine whereas their use in the Western hemisphere has only increased in the last decades. The International Journal of Medicinal Mushrooms, cited articles, books and reviews confirm the trend in medicinal mushrooms use. Mushrooms exhibit a multitude of beneficial biologically active compounds. Mushrooms are more widely consumed by people each day due to their nutritive and medicinal properties.

Belonging to taxonomic category of basidiomycetes or ascomycetes, mushrooms possess antioxidant and antimicrobial properties. Cacti are also considered one of the richest sources of neuroregenerative, anticancer and immunomodulating agents. Thus, these chemicals from mushrooms, cacti and in combination with botanicals, work symbiotically exemplifying a full spectrum of benefits that will pave the future with a novel alternative option in healthcare.

The pharmaceutical and beverage industries both involve products heavily marketed worldwide that carry harmful side effects. For example, energy boosted type soft drinks are especially popular and characteristically contain artificial sweeteners, caffeine, ephedra, taurine, guarana, phosphoric acid, preservatives, and a blend of flavorings. These soft drinks are not always attractive for the health conscious, especially for people suffering from medical conditions. Another popular market of beverages that have harmful side effects is that of alcohol beverages. Alcohol intake over periods of time often leads to serious health problems and often dependency, weight gain, high blood pressure, cardiovascular disease, liver disease, diabetes, anxiety disorders, etc. Many of these conditions warrant medical intervention and prescription care and can lead to death.

Due to the popularity of these beverages such as these, an improved nutritional profile for these markets is in demand. A beverage with fresh new flavors and an improved nutritional profile and without the negatives of empty calories, and added natural coloring is needed. It is desirable for consumers to have available a beverage having nutritional advantages for recreational use or for nutritive purpose. This creation would provide safe alternatives that avoid all the harmful side effects from alcohol, sodas, and energy drinks by avoiding harmful additives. By combining different strains of mushrooms, mycelia and botanicals and creating a symbiotic relation amongst ingredients. These products will pave the future for not only for the beverage industry but the pharmaceutical industry as well with a novel and new alternative option without harmful side effects as choices we have today.

### BRIEF SUMMARY OF THE INVENTION

The subject invention concerns a composition comprising, and method of producing, an edible, mushroom-producing fungi containing a significant amount of nutritional properties, to provide a nutritional or dietary supplement or pharmaceutical product for alleviating anxiety or stress in a person in need thereof.

The method comprises the steps of:
(a) supplying a growth environment comprising water liquid culture medium and a substrate comprising an organic grain for cultivation of edible mushroom-producing fungi;
(b) adding one or more nutritional enhancements to the growth environment;
(c) adding a spawn of at least one edible mushroom-producing fungi selected from the basidiomycetes or ascomycetes; and
(d) cultivating the edible mushroom-producing fungi in the growth environment for a sufficient time to permit the mushroom-producing fungi to develop nutritional significance.

The method can further comprise the step of:
(e) adding a dried or liquid botanical to the substrate.

The method can further comprise the steps of
(f) harvesting the cultivated mushroom-producing fungi,
(g) drying or liquefying the harvested mycelium-fungi, and
(h) extracting components from the dried or liquid mycelium fungi.

Preferably, the mycelium-fungi are dried using air heated to no great than 120° F.

The mycelia and mushroom producing fungi used in the described method are selected based on their chemical properties.

The subject invention further concerns an oral nutritional or dietary supplement, nutraceutical, or pharmaceutical composition for use in neuro-regeneration and stimulation of the immune system in an animal or human. The oral composition comprises:
(a) an orally ingestible carrier or diluent; and
(b) plurality of basidiomycetes or ascomycetes mushroom or mycelial ingredients provided in amounts as described herein.

The plurality of mushroom or mycelial ingredients are formed into a solid or liquid edible delivery system, comprising
(a) a multi mushroom compound comprising mycelial powder and mushroom extracts;
(b) an emulsifier;
(c) a vitamin/botanical mix comprising at least one of a mineral, enzyme, probiotic, prebiotics, or terpene;
(d) a flavoring; and
(e) optionally, a thickener.

An edible delivery system according to the invention can be prepared by the steps of:
(a) mixing a predetermined amount of liquid and a predetermined amount of a thickening agent to obtain a mixture;
(b) heating the mixture to get a gel-like consistency in either liquid to solid state;
(c) cooling the mixture;
(d) adding a predetermined amount of pharmaceutical or vitamin and botanical mixture;
(e) adding multi-mushroom component or extract; and
(f) further cooling for molding or packaging.

The method of preparing an edible delivery system can further comprise adding a pre-mixed multivitamin mix to the mixture, wherein the pre-mixed multivitamin mix is a vitamin mixture readily and commercially available. The multi-mushroom component or extract can be a pre-mixed mushroom extract, suitable mushroom-mycelial product, or tincture thereof, which are readily and commercially available. The method of preparing an edible delivery system can further comprise adding a coloring agent and a flavoring agent.

The above edible delivery system can be formulated as a solid, e.g., a troche or lozenge, table, chewable tablet, confection, or gummy. The edible delivery system can also be formulated in liquid form, as a beverage or drink. Alternatively, the delivery system can be formed as a suppository rather than an edible form.

Accordingly, the subject invention comprises a composition having nutritional or mood-enhancing properties when administered to a person in need thereof, the composition comprising a mushroom or extract thereof selected from the group consisting of Basidiomycota mushroom, Ascomycetes mushroom, and Sordariomycetes mushroom.

The composition can further comprise cactus or an extract thereof. One preferred source of cactus or cactus extract is Opuntia ficus-indica. Species of mushroom or cactus can include those selected from the group consisting of Hericium erinaceus, Tremella fuciformis, Agaricus blazei, Cordyceps sinensis, Cordyceps militaris, Flammulina velutipes, Ganoderma lucidum, Grifola frondosa, Letinula edodes, Phellenis linteus, Polyporus umbellatus, Inonotus obliquus, Pleurotus ostreatus, P. eryngii, P. citrinopileatus, P. djamor, Trametes versicolor, Volvariella volvacea, inonotus, Hypsizygus marmoreus, Pholiota nameko, Tuber melanosporum, Moschella hortensis, Bacopa Monnieri, Sarcodon spp., Antrodia camphorata, Pleurotus giganteus, Lignosus rhinocerotis, obliquus, Trametes versicolor, Poria cocos, and combinations thereof.

The composition can further comprise a cannabinoid, such as THC or CBD, or can include a botanical mixture.

One preferred embodiment of the composition can comprise:
0.15-50 wt % active ingredient compound and botanical mixture;
one or more, thickening or binding agent;
a stabilizer;
at least one fruit or vegetable juice and/or extract; and
a sweetener, enhancement, vitamins, mineral, amino acids, fruit, vegetable, herbal, essential oils/extracts, or a derivative thereof.

The above composition can be prepared as a solid dosage form or delivery system selected from a troche or lozenge, gummy, confection, tablet, or chewable tablet having 0.5-6 grams in weight.

A preferred embodiment of a liquid or beverage delivery system of the invention can comprise:
0.15-50 wt % active ingredient compound and botanical mixture;
one or more, thickening binding agents; stabilizers
at least one fruit or vegetable juice and/or extract;
a sweetener, and
additives or fortifiers selected from vitamins, mineral, amino acids, fruit vegetable herbal, essential oils/extracts, or a derivative thereof.

In a preferred embodiment the above composition can be prepared as a liquid beverage in a serving container having a volume of 0.2 ml to 480 ml.

The subject invention also concerns a method for treating a person in need of antioxidant, antimicrobial, anti-inflammatory neuroregenerative, anticancer, anti-epileptic, immunomodulating, antipsychotic, psychiatric, memory enhancement, cognitive function enhancement, performance enhancement, libido enhancement, or hydration treatment, comprising:
providing a composition comprising a mushroom or extract thereof, the mushroom or extract being selected from the group consisting of Basidiomycota mushroom, Ascomycetes mushroom, and Sordariomycetes mushroom; and
administering an effective amount of the composition to the person need such treatment.

Still further, the subject invention comprises a method for myceliation of an agricultural substrate for producing a composition comprising a mushroom or extract thereof from Basidiomycota mushroom, Ascomycetes mushroom, and Sordariomycetes mushroom. The resulting myceliation composition can have nutritional or mood-enhancing properties when administered to a person in need thereof. The myceliation method can comprise the steps of:
inoculating a fortified, agricultural grain substrate, in water or an aqueous medium with an aliquot of fungal culture selected from a group consisting of Basidiomycota and Ascomycota derived from spore, culture or liquid state fermentation to provide an optimized growth medium for fungal growth;
allowing mycelial growth under optimum growth conditions to obtain a myceliated substrate;
harvesting the myceliated substrate along with metabolized contents of the agricultural substrate after the mycelia growth reaches a desired stage; and
processing a live, liquid or dried myceliated substrate with one or more amendments to the medium which can be distilled water, spring water, filtered water, fruit or vegetable juice, broth, slurry, or gel, or infusion thereof, mixed or agitated with agar, gelatin, pectin, vegetable, grain, vegetable starch, nutritional yeast, malt extracts, dextrose, citric acid, ascorbic acid, honey, terpenes, cane sugar, essential oils, and combinations thereof.

The above myceliation method can be subjected to pre-processing steps and can include:
i. heat treatment comprising pasteurization, heat sterilization and filtration;
ii. optionally, providing a partial coating of a surface of substrate with at least one myceliation enhancing compound;
iii. a substrate known to exhibit neuroregenerative and neuroprotective properties.

A beverage product of the subject invention advantageously providing beneficial properties to a person ingesting the beverage, can be prepared by the steps of:
a) admixing Saw Palmetto honey, wood, seeds, bark, botanicals in a sanitized mason jar and place the mason jar with admixture into a 110-degree water bath for 12 days to achieve a decarboxylation process required to activate THC;
b) adding to the admixture from step a), 1 part of a floral tea damiana to 3 parts water and place in a 110-degree water bath for an additional 12 hours;
c) straining the composition to remove the solid components.

### DETAILED DESCRIPTION OF THE INVENTION

The present invention relates to a formulation and delivery system for a multi-mushroom mycelial compound and botanical mixture. Specifically, a solid, confection, or confection like, or gummy delivery system including an active ingredient composed of a mycelial extract compound and/or a mixture of cacti and botanicals. This invention further relates to liquid products, specifically to an infused beverage containing botanicals. These compositions can provide nutritional or mood enhancing characteristics for the benefit of a person in need thereof.

In principle, whole mushrooms, mycelium, extracts (from fruiting bodies or mycelium), and isolated compounds are suitable for use and are to be created into a confection/gummy/candy like multivitamin/supplement/nutraceutical delivery system. The delivery system can work synergistically with the mushroom mycelia extracts and or compound(s) within those extracts.

Additionally, cacti are or extracts thereof can be introduced as an ingredient for their beneficial properties with whole cacti, extracts, and isolated compounds suitable for use being similarly created into a confection/gummy/candy like multivitamin/supplement/nutraceutical delivery system. The delivery system can work synergistically with the mushroom, mycelial, or cactus extracts or compound(s).

The ingredients in the subject composition can be obtained by collection from the wild, organic cultivation, local foraging, as well as local farmed ingredients from the local farming community. The combination of ingredients with outsourced essential oils and extracts are handpicked and utilized to create flavor profiles to create a symbiotic relationship among ingredients.

Basidiomycota, ascomycetes, or Sordariomycetes classifications are to be prepared by extraction of mushrooms (dried or fresh) using heat or cold extraction methods with suitable solvents. Pure compounds are obtained by isolation from the natural or cultivated material or by chemical synthesis of fruiting bodies, mycelium, in fermentation, with liquid or solid substrates.

The pure compounds are to be medicinally enhanced based on conditions and or symptoms to be treated. The pure compounds are introduced as the active ingredient into a solid or liquid delivery system which can be savory food/confection/gummy/candy like multivitamin/supplement/nutraceutical/pharmaceutical/suppository.

The present invention further provides carrier dietary supplements that contain acceptable food supplements and food additives cacti and botanicals, to further provide a wide spectrum and valuable source of natural medicine for natural and or pharmaceutical treatment or supplementation for psychiatric treatment as well as memory and cognition functions.

Additionally, the present invention further provides a carrier dietary supplements that contain acceptable food supplements and food additives mushroom extract, mycelium, botanicals, to further provide a wide spectrum, valuable source of natural medicine for natural and or pharmaceutical treatment or supplementation for psychiatric treatment as well as memory and cognition functions.

The confection/gummy/candy like multivitamin/supplement/nutraceutical delivery system will provide a full spectrum of benefits and will contain a mushroom mycelia compound, supplementation and or pharmaceuticals boasting antioxidant, antimicrobial, anti-inflammatory neuroregenerative, anticancer and immunomodulating properties to manage, prevent, treat and or to alleviate symptoms related to malnutrition, infection, virus, disease.

The confection/gummy/candy like multivitamin/supplement/nutraceutical delivery system will provide a full spectrum of benefits and will contain Cacti, supplementation and or pharmaceuticals boasting antioxidant, antimicrobial, anti-inflammatory neuroregenerative, anticancer and immunomodulating properties to manage, prevent, treat and or to alleviate symptoms related to malnutrition, infection, virus, disease.

The delivery system however is not limited to the confection/gummy/candy like multivitamin/supplement/nutraceutical delivery system but also includes a beverage delivery system. In principle, whole mushrooms, mycelium, extracts (from fruiting bodies or mycelium) and isolated compounds are suitable for use and are to be created into a liquid multivitamin/supplement/nutraceutical delivery system. The beverage delivery system works synergistically with the mushroom mycelia extracts and or compound/s as in the ingredients have beneficial property; the beneficial properties may be enhanced through organic cultivation; organic measures may be essential in a circumstance when treating a sensitive medical conditions but not deemed totally necessary in example of a recreational product.

In principle, whole mushrooms, cacti, botanicals, mycelium, extracts (from fruiting bodies or mycelium) and isolated compounds are suitable for use and are to be created into products for use. The multivitamin/supplement/nutraceutical/pharmaceutical/recreational use compound is to be entered into a beverage delivery system. Mushrooms, mycelia, cacti, and other botanicals may be cultivated or outsourced can be in whole, liquid, or powder forms or as extracts.

The materials could be obtained by collection from the wild, cultivation, shelf bought, outsourced, and or inorganic or organic. This will be based on the availability of the product, customer, and demand of market as well as patient profiles and can be introduced based on desired effect, beneficial property and or mood enhancing attributes.

The compositions of the invention can also include Cannabis, hemp, CBD, or an entourage preparation that includes terpenoids and may include phytocannabinoids, phenylpropanoids or other compounds that contribute to flavor, aroma, and the effect of mushroom mycelial property and phytocannabinoids in the preparation.

The infused liquid will provide a full spectrum of benefits and will contain a mushroom and or mycelia compound, may or may not contain Cannabis, Hemp CBD Product.

The infused liquid composition of the invention can provide a full spectrum of benefits from combination of ingredients and or from within organic cultivation techniques; boasting antioxidant, antimicrobial, anti-inflammatory neuroregenerative, anticancer and immunomodulating properties valuable source of natural medicine supplementation and or pharmaceutical treatment for psychiatric treatment as well as memory and cognition function, enhance performance, libido, and aid dehydration in the form of a recreational beverage.

The pure compounds and liquid delivery system are to be medicinally enhanced based on conditions and or symptoms to be treated; the pure compounds are introduced as the active ingredient into a liquid delivery system which can be savory or confection like.

The infused liquid can be savory, flavorless, sweet, spicy, herbal, bitter, floral, tropical, or smokey. The infused liquid can be in the form of a multivitamin, a dietary supplement, a nutraceutical, a pharmaceutical, or a recreational use product.

It is therefore an object of the present invention to provide a nutritionally enhanced infused botanical mixture, to be utilized as a beverage containing a recreational and or supplementation use for which the delivery method may be a sipping cocktail to create the effect of drinking an alcoholic beverage. It is a further object of the present invention to provide a non-alcoholic liquor, liqueur, Tonic, Mixer, and/or bitters that can be further added to create mixed beverages.

It is a further object of the present invention to provide a delivery method that is beverage such as a juice drink, carbonated beverage or soda, beer, energy drink, intravenous solution, topical substance, tonic, tea, and/or slush. Sweeteners, flavors, additives, and carbonation can also be added as desired.

A further object of the present invention is to provide an energy drink, which in addition to the thirst quenching effect has an invigorating and energizing effect on the body, provides the body with sufficient vitamins and nutrients, relieves hangovers, relieves thirst, and can promote health with efficacy.

Another object of the present invention is to provide a beverage, which in addition to supplementation, can provide the body with sufficient vitamins and nutrients for recreational and or pharmaceutical use. It is another object of the present invention is for the active ingredient to work synergistically with delivery system and to be presented as an infused beverage.

The infused liquid delivery system is created based on the symptoms of condition(s) and specific active ingredients of the compound, a singular strain dried/extracted mycelia, botanical mixture, vitamin mix, dietary supplement, amino acid, probiotic, prebiotic and/or pharmaceutical, that may or may not be a beverage. The delivery method may be oral, topical, or an intravenous solution.

The constitution of the composition of the present invention can vary to a certain extent depending upon the provided market, consumer desired effects, and or patient demands/requirements.

In one embodiment, the present invention provides a method of solid state myceliation of an agricultural substrate for producing functional composition by inoculating the agricultural substrate with pure strains of fungal culture selected from a group consisting of Basidiomycota and Ascomycota. Species include but are not limited to Hericium erinaceus, Tremella fuciformis, Grifola frondosa, Agaricus blazei, Cordyceps sinensis, Cordyceps militaris, Flammulina velutipes, Ganoderma lucidum, Grifola frondosa, Letinula edodes, Phellenis linteus, Polyporus umbellatus, Inonotus obliquus, Pleurotus ostreatus, P. eryngii, P. citrinopileatus, P. djamor, Trametes versicolor, Volvariella volvacea, inonotus, Hypsizygus marmoreus, Pholiota nameko, Tuber melanosporum, Moschella hortensis, Bacopa Monnieri, Sarcodon spp., Antrodia camphorate, Pleurotus giganteus, Lignosus rhinocerotis, obliquus, Trametes versicolor, Poria cocos, and combinations thereof.

In one embodiment, the present invention pertains to methods of liquid state and solid state myceliation of organic agricultural substrates, involving inoculation, fermentation through cultivation producing a compound of enhanced health benefits and nutritional profile.

The present invention further pertains to a method of liquid state and solid state myceliation of agricultural substrates to be further processed for consumption, wherein the method comprises the steps of:
a. inoculating a fortified, agricultural grain substrate, in water or an aqueous medium with an aliquot of fungal culture selected from a group consisting of Basidiomycota and Ascomycota derived from spore, culture or liquid state fermentation to provide an optimized growth medium for fungal growth;
b. allowing mycelial growth under optimum growth conditions to obtain a myceliated substrate;
c. harvesting the myceliated substrate along with metabolized contents of the agricultural grain substrate after the mycelia growth reaches a desired stage; and
d. processing a live or dried myceliated substrate with one or more amendments to the medium which can be distilled water, spring water, filtered water, fruit or vegetable juice, broth, slurry, or gel, or infusion thereof, mixed or agitated with agar, gelatin, pectin, vegetable, grain, vegetable starch, nutritional yeast, malt extracts, dextrose, citric acid, ascorbic acid, honey, terpenes, cane sugar, essential oils, and combinations thereof.

In an embodiment, agricultural substrates of the present invention can be pre-processed before inoculation. Pre-processing depends on the nature of substrate and can include:
i. heat treatment comprising pasteurization, heat sterilization and filtration; and/or
ii. partial coating of a surface of substrate with at least one myceliation enhancing compound;
iii. at least one substance known to exhibit neuroregenerative and neuroprotective properties.

In an embodiment, a step of pre-processing comprises heat treatment based on nature of the substrate in order to eradicate or minimize microbial load present on its surface. For example, agricultural substrates such as grains, cereals, and seeds prior to inoculation with pure culture of fungal species are subjected to heat sterilization treatment comprising pressure cooking for 90 minutes at 15 lb/inch square in order to remove microbial content present on the substrate without adversely affecting its taste and nutritional quality.

In another embodiment, heat treatment pre-processing comprises pasteurization. For example, agricultural substrates such as Peat, cocofiber, manure, straw, vermiculite, coffee herbs, spices, dry beans, seeds grain are pasteurized being subject to dry/steam/liquid heat treatment at 145 degrees F. to 250 degrees F. for 30 to 90 minutes.

In an embodiment, pre-processing can also comprise filtration of a substrate in liquid form. For example, substrate such as aqueous solution of any substrate can be subjected to filtration in order to remove microbial content prior to inoculation with pure culture of fungal species.

In accordance with the present invention, agricultural substrate in example as in rice, seed, corn, beans, the media substrate may be pre-processed before inoculation by at least partially coating a surface of the substrate with a least myceliation enhancing compound such as an anti-caking agent. For food-quality products, the anti-caking agent is edible, for example coconut, avocado oil can be used to coat the substrate to improve myceliation and neuro-regenerative properties.

In another embodiment, pre-processing can also comprise filtration of a substrate in liquid form. For example, substrate such as aqueous solution of any substrate can be subjected to filtration in order to remove microbial content prior to inoculation with pure culture of fungal species.

The present invention relates to a method of myceliation of agricultural substrates for producing health benefits, wherein the method comprises of inoculating an agricultural substrate with one or more species of pure fungal culture comprising Basidiomycota and Ascomycota species derived from liquid state fermentation, enabling mycelial growth on the agricultural substrate by controlling growth conditions and harvesting of a myceliated agricultural product after the mycelial growth reaches a desired stage.

In an embodiment, a desired stage of mycelia growth can be reached, for example, when fruiting bodies begin to appear on a surface of an agricultural substrate. Mycelial growth reaching a desired stage can also be determined based on resistance offered by the liquid media against stirring or mixing of its contents. Mycelial growth reaching a desired stage can also be determined by effects desired for the compound delivered in a confection/gummy/candy like supplement/nutraceutical to boast superlative neuroregenerative properties.

In other embodiments of the invention, lighting specifications, air flow, temperature, and other data can be controlled. In one embodiment, in accordance with the present invention, myceliation of a solid agricultural substrate or aqueous solution. The solid agricultural substrate or solution compromised of Agar, bloodmeal, bone meal, Peet moss, fish emulsion, shells, crickets, worms, oyster shell, cereals, grains, species of wheat, rye, brown rice, white rice, red rice, wild rice, basmati rice, barley, triticale, rice, sorghum, oats, millets, quinoa, buckwheat, wood chips/shavings/pellets, logs, trees.

The solid agricultural substrate or aqueous solution are enhanced with amendments. The amendments are further noted within patent as botanical mixture. The botanical mixture works synergistically to enhance microbial property of growing medium within propagation. The botanical mixture is further referenced and utilized within steps of creating the delivery system.

In one embodiment in accordance with the present invention, further additions to the agricultural substrate can consist of media combinations within cultivation and may be specifically created per circumstance to enhance active ingredients selected from the group comprising one or more combinations of Argyreia nervosa, blood meal, fish emulsion, oyster shell, cereals, grains, species of wheat, rye, brown rice, white rice, red rice, wild rice, basmati rice, barley, triticale, rice, sorghum, oats, millets, quinoa, buckwheat, fonio, farroh amaranth, teff, durum, apples, pears, apricots, cherries, almonds, peaches, strawberries, raisins, manioc, cocoa, banana, Rubiaceae sp. (coffee), lemons, oranges and grapefruit; tomatoes, potatoes, peppers, eggplant, Allspice, mango powder, mango butter derived from seed, Angelica, Anise (Pimpinella anisum), Aniseed myrtle (Syzygium anisatum), Annatto (Bixa orellana), banana, banana leaf, Apple mint (Mentha suaveolens), Artemisia vulgaris, Mugwort, Asafoetida (Ferula assafoetida), Bacopa species (Bacopa monnieri), Berberis, Banana, Basil (Ocimum basilicum), Bay leaves, Bistort (Persicaria bistorta), Black cardamom, Black cumin, Blackcurrant, Black limes, Bladder wrack (Fucus vesiculosus), Blue Cohosh, Blue-leaved Mallee (Eucalyptus polybractea), Bog Labrador Tea (Rhododendron groenlandicum), Boldo (Peumus boldus), Bolivian Coriander (Porophyllum ruderale), Borage (Borago officinalis), Calamus, Calendula, Calumba (Jateorhiza calumba), Chamomile, Cruciferous vegetation, Cannabis and Hemp (Cannabis sativa, C. indica and C. ruderalis) (Decarboxelated, Dried) Candied flowers, paper (Capparis spinosa), Caraway, Cardamom, Carob Pod, Cacti, Cassia, Casuarina, Catnip, Cat's Claw, Catsear, caspacian, Cayenne pepper, Celastrus Paniculatus, Comfrey, Celery salt, Celery seed, Centaury, Chervil (Anthriscus cerefolium), Chickweed, Chicory, Chile pepper, Chili powder, Cinchona, Chives (Allium schoenoprasum), Cicely (Myrrhis odorata), Cilantro (see Coriander) (Coriandrum sativum), Cinnamon (and Cassia), and bark, Cinnamon Myrtle (Backhousia myrtifolia), Ceylon Cinnamon, Clary, Cleavers, Clover, Cloves, Coltsfoot, Comfrey, Common Rue, Condurango, Coptis, Coriander, Costmary (Tanacetum balsamita), Couchgrass, Cocoa, Cacao, cocoa butter, Cow Parsley (Anthriscus sylvestris), Cowslip, Cramp Bark (Viburnum opulus), Cress, Cuban Oregano (Plectranthus amboinicus), Cudweed, Cumin, Curry leaf (Murraya koenigii), Damiana (Turnera aphrodisiaca), Dandelion (Taraxacum officinale), Demulcent, Devil's claw (Harpagophytum procumbens), Dill seed, Dill (Anethum graveolens), Dorrigo Pepper (Tasmannia stipitata), Echinacea, Echinopanax Elatum, Edelweiss, Elderberry, Elderflower, Elecampane, Eleutherococcus senticosus, Epazote (Chenopodium ambrosioides), Ephedra, Eryngium foetidum, Eucalyptus, Fennel (Foeniculum vulgare), Fenugreek, Feverfew, Figwort, Five-spice powder (Chinese), Fo-ti-tieng, Fumitory, Galangal, Garam masala, Garden cress, Garlic chives, Garlic, Ginger (Zingiber officinale), Ginkgo biloba, Ginseng, Gotu kola (Centella asiatica). Siberian (Eleutherococcus senticosus), Goat's Rue (Galega officinalis), Goada masala, Golden Rod, Golden Seal, Gotu Kola, Grains of paradise (Aframomum melegueta), Grains of Selim (Xylopia aethiopica), Grape seed extract, Green tea, (Camellia sinensis) Ground Ivy, Guaco, Gypsywort, Ginger (Zingiber officinale), Holy Basil (Ocimum sanctum), Hu Zhang (Polygonum cuspidatum), Hawthorn (Crataegus sanguines), Hawthorne Tree, Hemp, Herbes de Provence, Hibiscus, Holly, Holy Thistle, Honey, Hops, Horehound, Horseradish, Horsetail (Equisetum telmateia), Hyssop (Hyssopus officinalis), Jalap, Jasmine, Jiaogulan (Gynostemma pentaphyllum), Joe Pye weed (Gravelroot), John the Conqueror, Juniper, Kaffir Lime Leaves (Citrus hystrix, C. papedia), Kaala masala, Knotweed, Kokam, Labrador tea, Lady's Bedstraw, Lady's Mantle, Land cress, Lavender (Lavandula spp.), lavender (Lavandula spica and related species in the genus Lavandula), Ledum, Lemon Balm (Melissa Officinalis), Lemon basil, Lemongrass (Cymbopogon citratus, C. flexuosus, and other species), Lemon Ironbark (Eucalyptus staigeriana), Lemon mint, Lemon Myrtle (Backhousia citriodora), Lemon Thyme, Lemon verbena (Lippia citriodora), Licorice-adaptogen, Lime Flower, Limnophila aromatica, Lingzhi, Linseed, Liquorice, Long pepper, Lovage (Levisticum officinale), Luohanguo, Mace, Mahlab, Malabathrum, Manchurian Thorn Tree (Aralia manchurica), Mandrake, Marjoram (Origanum majorana), Marrubium vulgare, Marsh Labrador Tea, Marshmallow, Mastic, Meadowsweet, Mei Yen, Melegueta pepper (Aframomum melegueta), Mint, Milk thistle (Silybum), Bergamot (Monarda didyma), morning glory (Ipomoea tricolor and related species) and seeds of) Motherwort, Mountain Skullcap, Mullein (Verbascum thapsus), Mustard, Mustard seed, Nashia inaguensis, Neem, Nepeta, Nettle, Nigella sativa, Kolanji, Black caraway, Noni, Nutmeg, Mace, Marijuana, Oenothera (Oenothera biennis), Olida (Eucalyptus olida), Oregano (Origanum vulgare, O. heracleoticum), Orris root, Osmorhiza, Olive Leaf (used in tea and as herbal supplement), Panax quinquefolius, Pandan leaf, Paprika, Parsley (Petroselinum crispum), Passion Flower, Patchouli, Pennyroyal, Pepper (black, white, and green), Peppermint, Peppermint Gum (Eucalyptus dives), Perilla, Plantain, Pomegranate, Ponch phoran, Poppy seed, Primrose (Primula), Psychotria species (Psychotria viridis, Wild Coffee), (, and plants containing ibogaine (Tabernanthe iboga, Voacanga africana and Tabernaemontana undulate), peyote (Lophophora williamsii), dry tea mixes, Psyllium, Purslane, Quassia, Quatre epices, Ramsons, Raspberry (leaves), Restharrow, Rhodiola rosea, Riberry (Syzygium luehmannii), Rocket/Arugula, Roman chamomile, Rooibos, Rosehips, Rosemary (Rosmarinus officinalis), Rosmarinus eriocalyx, species in the genus Rosmarinus), Rowan Berries, Rue, Safflower, Saffron, Sage, Saw Palmetto, skullcap (Scutellaria lateriflora) Moreover, Salvia divinorum, aka Diviner's Sage, ayahuasca caapi (Salvia officinalis), Saigon Cinnamon, St John's Wort, Salad Burnet (Sanguisorba minor or Poterium sanguisorba), Salvia, Sichuan Pepper (Sansho), saw palmetto, Sassafras, Savory (Satureja hortensis, S. montana), Schisandra (Schisandra chinensis), Scutellaria costaricana, Senna (herb), Senna obtusifolia, Sesame seed, Sheep Sorrel, Shepherd's Purse, Sialagogue, Siberian Chaga, Siberian ginseng (Eleutherococcus senticosus), Siraitia grosvenorii (luohanguo), Skullcap, Sloe Berries, Smudge Stick, Sonchus, Sorrel (Rumex spp.), Southernwood, Spearmint, Speedwell, Squill, Star anise, Stevia, Strawberry Leaves, Suma (Pfaffia paniculata), Sumac, Summer savory, Sutherlandia frutescens, Sweet grass, Sweet cicely (Myrrhis odorata), Sweet woodruff, Szechuan pepper (Xanthoxylum piperitum), San Pedro, trichocereus pachanoi Tacamahac, Tamarind, Tandoori masala, Tansy, Tarragon (Artemisia dracunculus), Tea, Teucrium polium, That basil, Thistle, Thyme, Toor Dall, Tormentil, Tribulus terrestris, Tulsi (Ocimum tenuiflorum), Turmeric (Curcuma longa), Uva Ursi also known as Bearberry, Vanilla (Vanilla planifolia), Vasaka, Vervain, Vetiver, Vietnamese Coriander (Persicaria odorata), Wasabi (Wasabia japonica), Watercress, Wattleseed, Wild ginger, Wild Lettuce, Wild thyme, Winter savory, Witch Hazel, Wolfberry, Wood Avens, Wood Betony, Woodruff, Hawaiian baby woodrose, Wormwood, Yarrow, Yerba Buena, Yohimbe, Za'atar, Zedoary Root, or derivations thereof of seed, bark, flower, stem, trim fruited body, solid form, extract, gaseous, aqueous or semi-aqueous solution can be fermented, thereof not limited to and to be further updated within study of the patent.

In an embodiment, optimum growth conditions for mycelial growth can be controlled by controlling parameters such as selective pH, temperature, humidity, incubation period, media composition, aeration, and pressure among such other parameters.

The semi-anaerobic environment can be created by depositing the substrate in an evacuated container or partially evacuated container. In a further embodiment, evacuation of oxygen or air from the container's holding the substrate creates a semi-anaerobic environment. In an alternative embodiment, the container is capped to eliminate (or reduce) airflow from outside of the container/room.

The lighting, aeration, i.e. flow of air, can be regulated according to measured mycelium growth where initial conditions require a minimal amount of light and air flow. In this way an optimal rate of mycelium growth is achieved.

In one embodiment, not limited to using myceliation methods as disclosed herein, agricultural substrates myceliated with Basidiomycota and Ascomycota classifications can be deep-frozen for later use after fermentation in either small-scale or large-scale solid-state production, wherein a fermented product can then be cooked, fried, boiled, mashed, pressed, baked, toasted, roasted, or blanched in aqueous solution after completion of the fermentation. Products may be used directly, and/or reformulated into value-added compounds.

Active ingredient can be formulated from stationary liquid tissue culture or from a blended mycelial culture of agricultural substrate. Using liquid tissue broth to create mushroom concentrates for formulation of cultures grown on solidified/gelled, aqueous mediums, mature cultures grown on substrate (i.e. grain) can include fruit body tissue, primordial tissue, and spores, encompassing all four stages of the mushroom's life cycle.

Preferred drying methods include freeze drying, air drying, spray drying and drum drying and the methods and apparatus for drying mycelium. Extracts are preferably extracted from living mycelium.

The present invention provides a myceliated agricultural product enriched with nutrients for consumption, to be further processed into a pharmaceutical and or supplement which can be functional food or liquid. Proper cultivation techniques are performed to reach desired stage(s) in development of the mycelia/mushroom matter to be collected utilized fresh or dried to be further processed into compound.

In one embodiment, the present invention pertains to methods of liquid-state and solid-state myceliated substance to be further introduced into a compound.

In one embodiment to create compound, decoction of plant material can be achieved by adding boiling water and mushrooms of slow-cooking in a slow boil/simmer reduction. decocting the marc pressed from the tincture in a 1:5 marc/spring water ratio, simmering, pressing and composting the decocted marc, and then simmering the water extract further until it has evaporated to 1/10 of its original volume.

In one embodiment to provide pure water soluble compounds mycelia, primordial tissue fruiting bodies can be dried, ground and directly added.

In one embodiment of this invention extraction temperatures can vary cold, water, hot water, dual and vary in steps, providing an extract that can be amended into compound.

The present invention pertains to methods of liquid-state and solid-state myceliated substance processed into a multi mushroom compound to be further introduced active ingredient.

The active ingredient can vary in measurement, amounts, ingredients, and each are reported as having pharmacological activity, such as neuroregeneration, immunological, inflammation, detoxification, anti-cancer effects.

It is another object of the present invention is for the active ingredient to work synergistically with delivery system.

The present invention further relates to oral delivery systems for active ingredient. The delivery system is created based on the symptoms of condition(s) specific active ingredient compound, a singular strain dried/extracted mycelium, botanical mixture, and/or pharmaceutical.

The delivery system can vary in measurement, amounts, ingredients, and each are reported as having pharmacological activity, such as neuroregeneration, immunological, inflammation, detoxification, anti-cancer effects, and could provide nutritional supplementation. Creating a full spectrum of beneficial properties.

Combining mushrooms and mycelia with botanicals for example Aloe vera in the cacti family presents additional digestive benefits, such as reducing the incidence of stomach ulcers and improving digestion. The many anti-inflammatory compounds in aloe vera juice, such as vitamin C, prove essential.

To further provide a full spectrum of benefits of plant medicines when botanicals and fungi are combined, create essential neuro-medicine. The delivery system for the active ingredient further enhanced with a botanic mixture.

When cacti are introduced to the botanical mixtures, growth environment, delivery system and components herein. Cacti traditionally provide valuable health supporting nutrients and have applications within the pharmaceutical industry. Cactus with number of uses has immense potential to be the food of future. For example, the nopales have been used in traditional medicine for disease treatment and prevention. In proven studies cactus plants have been used to treat: neurological disorders, migraines, glaucoma, wounds, fatigue, liver conditions, and ulcers.

For example, purported benefits of the nopal include lowering blood sugar, healing wounds, and lowering cholesterol. Studies have also confirmed antioxidant and anti-inflammatory properties.

In a further example, Magnolia bark has been used in Chinese medicine for more than 2,000 years. This herb is aromatic, pungent, warming and stimulating and strengthening digestive system, containing antiseptic, antifungal, antibacterial, anti-inflammatory, and antispasmodic properties and may be used to treat various inflammatory conditions, colds, and flu.

In another example, honey offers anti allergen, antibacterial activity and immunomodulatory property, beneficial enzymes medical care and within agriculture promote root growth in plants and provide microbial nutrients Saw palmetto honey and Manuka honey have been reported to exhibit antimicrobial activity against pathogenic bacteria such as Staphylococcus aureus (S. aureus) and Helicobacter pylori (H. pylori) content in enzymes, antioxidants, vitamins, acids, fitonutrients.

The botanic mixture created can be from juice, extract, dried, flower, seed, root, trim, leaf, bud, flower or derivatives of ayurvedic plants, Aloe, Cacti, Cannabis, CBD, coconut, Cruciferious vegetation, fruit, botanicals, herbs, peels, skins, seeds, roots, stems, leaves, flowers, apples and pears, apricots, cherries, almonds, peaches, strawberries, raisins, coffee, lemons, oranges and grapefruit; tomatoes, potatoes, peppers, eggplant, African blue basil, Angelica, Anise (Pimpinella anisum), Aniseed myrtle (Syzygium anisatum), Annatto (Bixa orellana), Apple mint (Mentha suaveolens), Artemisia vulgaris, Mugwort, Asafoetida (Ferula assafoetida), Ajwain (Trachyspermum ammi), Allspice (Pimenta dioica), Amchur—mango powder (Mangifera) Angelica (Angelica archangelica) Anise (Pimpinella anisum) arrowroot, Arugula/Rocket, Berberis, Banana, Barley, Basil (Ocimum basilicum), Bay leaves, Baobob, Bistort (Persicaria bistorta), Black cardamom, Black cumin, Blackcurrant, Black limes, Bladder wrack (Fucus vesiculosus), Blue Cohosh, Blue-leaved Mallee (Eucalyptus polybractea), Bog Labrador Tea (Rhododendron groenlandicum), Boldo (Peumus boldus), Bolivian Coriander (Porophyllum ruderale), Borage (Borago officinalis), Berberis, Banana, Basil (Ocimum basilicum), Bay leaves, Bistort (Persicaria bistorta”), Boldo (Peumus boldus), Borage (Borago officinalis), Cacti species, Calamus, Candlenut Calendula, Calumba (Jateorhiza calumba), Chamomile, Cruciferous vegetation, Candied flowers, Caper (Capparis spinosa), Caraway, Cardamom, Carob Pod, Cassia, Casuarina, Catnip, Cat's Claw, Catsear, caspacian, Cayenne pepper, Celastrus Paniculatus, Comfrey, Celery, Celery seed, Centaury, Chervil (Anthriscus cerefolium), Chickweed, Chicory, Chile pepper, Chili powder, Cinchona, Chives (Allium schoenoprasum), Cicely (Myrrhis odorata), Cilantro (see Coriander) (Coriandrum sativum), Cinnamon (and Cassia), and bark, Cinnamon Myrtle (Backhousia myrtifolia), Ceylon Cinnamon, Clary, Cleavers, Clover, Cloves, Coltsfoot, Comfrey, Common Rue, Condurango, Coptis, Coriander, Costmary (Tanacetum balsamita), Couchgrass, coffee, Cocoa, Cacao, cocoa butter, Cola Nitida acuminata, Cow Parsley (Anthriscus sylvestris), Cowslip, Cramp Bark (Viburnum opulus), Cress, Cuban Oregano (Plectranthus amboinicus), Cudweed, Cumin, Curry leaf (Murraya koenigii), Centaury, Chervil (Anthriscus cerefolium), Damiana (Turnera aphrodisiaca), Dandelion (Taraxacum officinale), Demulcent, Devil's claw (Harpagophytum procumbens), Dill seed, Dill (Anethum graveolens), Dorrigo Pepper (Tasmannia stipitata), Echinacea, Echinopanax Elatum, Edelweiss, Elderberry, Elderflower, Elecampane, Eleutherococcus senticosus, Epazote (Chenopodium ambrosioides), Ephedra, Eryngium foetidum, Eucalyptus, Fennel (Foeniculum vulgare), Fenugreek, Feverfew, Figwort, Five-spice powder (Chinese), Emmenagogue, Epazote (Chenopodium ambrosioides), Ephedra—Eryngium foetidum Eucalyptus, Eyebright, Fennel (Foeniculum vulgare), Fenugreek, Feverfew, Figwort, File powder, Fo-ti-tieng, Fumitory, Galangal, Garam masala, Garden cress, Garlic chives, Garlic, Ginger, Gideon, Ginkgo biloba, Ginseng, Ginseng, Siberian (Eleutherococcus senticosus), Gentian, Goat's Rue (Galega officinalis), Goada masala, Golden Rod, Golden Seal, Gotu Kola, Grains of paradise, Grape seed extract, Green tea, Ground Ivy, Guaco, Gypsywort, Hawthorn (Crataegus sanguinea), Hawthorne Tree, Hemp, Herbes de Provence, Hibiscus, Holly, Holy Thistle, Hops, Horehound, Horseradish, Horsetail (Equisetum telmateia), Hyssop (Hyssopus officinalis) Jalap, Jasmine, Jiaogulan (Gynostemma pentaphyllum), John the Conqueror, Juniper, Kaffir Lime Leaves (Citrus hystrix, C. papedia), Kaala masala, Knotweed. Kokam, Labrador tea, Lady's Bedstraw, Lady's Mantle, Land cress, Lavender (Lavandula spp.), Ledum, Lemon Balm (Melissa Officinalis), Lemon basil, Lemongrass (Cymbopogon citratus, C. flexuosus, and other species), Lemon Ironbark (Eucalyptus staigeriana), Lemon mint, Lemon Myrtle (Backhousia citriodora) Lemon Thyme, Lemon verbena (Lippia citriodora), Licorice—adaptogen, Lime Flower, Limnophila aromatica, Lingzhi, Linseed, Liquorice, Long pepper, Lovage (Levisticum officinale), Luohanguo, magic berry, Mace, Mahlab, Malabathrum, Manchurian Thorn Tree (Aralia manchurica), Mandrake, Marijuana, Marjoram (Origanum majorana) Marrubium vulgare, Marsh Labrador Tea, Magnolia, Marshmallow, Mastic, Meadowsweet, Mei Yen, Mint (Mentha spp.), Milk thistle (Silybum), Bergamot (Monarda didyma), Motherwort, Mountain Skullcap, Mullien (Verbascum thapsus), Mustard, Mustard seed, Nashia inaguensis, Neem, Nepeta, Nettle, Nigella sativa, Nigella (Kolanji, Black caraway), Noni, Nutmeg (and Mace), Oenothera (Oenothera biennis et al), Olida (Eucalyptus olida), Oregano (Origanum vulgare, O. heracleoticum, and other species), Orris root, Osmorhiza, Olive Leaf (used in tea and as herbal supplement), Pea flower Clitoria ternatea, Panax quinquefolius, Pandan leaf, Paprika, Parsley (Petroselinum crispum), Passion Flower, Patchouli, Pennyroyal, Pepper (black, white, and green), Peppermint Gum (Eucalyptus dives), Perilla, Plantain, Pomegranate, Ponch phoran, Poppy seed Primrose (Primula) candied flowers, tea, Psyllium, Purslane, Quassia, Quatre épices, Ramsons Ras el-hanout, Raspberry (leaves), Reishi, Restharrow, Rhodiola Rosea, Riberry (Syzygium luehmannii) Roman chamomile, Rooibos, Rosehips, Rosemary (Rosmarinus officinalis) Rowan Berries, Rue, Safflower, Saffron, Sage (Salvia officinalis), Saigon Cinnamon, St John's Wort, Salad Burnet (Sanguisorba minor or Poterium sanguisorba), Salvia, Sichuan Pepper (Sansho), Sassafras, Savory (Satureja hortensis, S. montana), Schisandra (Schisandra chinensis), Scutellaria costaricana, Senna (herb), Sea moss, Senna obtusifolia, Sesame seed, Sheep Sorrel, Shepherd's Purse, Sialagogue, Siberian Chaga, Siberian ginseng (Eleutherococcus senticosus), Siraitia grosvenorii (luohanguo), Skullcap, Sloe Berries, Sonchus, Sorrel (Rumex spp.), Southernwood, Spearmint, Speedwell, Spilanthes Toothache Plant, Squill, Succulents, Star anise, Stevia, Strawberry Leaves, Suma (Pfaffia paniculata), Sumac, Summer savory, Sutherlandia frutescens, Sweet grass, Sweet cicely (Myrrhis odorata), Sweet woodruff, Szechuan pepper (Xanthoxylum piperitum), Tacamahac Tamarind, Tansy, Tarragon (Artemisia dracunculus), Tea, Teucrium polium, Thai basil, Thistle, Thyme, Toor Dall, Tormentil, Tribulus terrestris, Tulsi (Ocimum tenuiflorum), Turmeric, Uva Ursi also known as Bearberry, Vanilla (Vanilla planifolia), Vasaka, Vervain, Vetiver, Vietnamese Coriander, Wasabi, Watercress, Watermelon seed. Wattleseed, Wild ginger, Wild Lettuce, Wild thyme, Winter savory, Witch Hazel, Wolfberry, Wood Avens, Wood Betony, Woodruff, Wormwood, Yarrow, Yerba Buena, Yerba Mate, Yohimbe, Za'atar, Zedoary Root.

In one embodiment the delivery system can be created by mixing a predetermined amount of liquid and a predetermined amount of a thickening agent to obtain a mixture. The mixture can be heated/boiled to get a gel-like consistency. The mixture is cooled to specific temperature. A predetermined amount of active ingredient compound, botanical mixture; pharmaceuticals vitamins, minerals, amino acid, enzymes, terpene, are then added. The mixture is in a solid confection or confection like form.

In one embodiment the delivery system can be created by mixing a predetermined amount of liquid a predetermined amount of a thickening agent to obtain a mixture. The mixture can be heated/boiled to get a gel-like consistency. The mixture is cooled to specific temperature. A predetermined amount of active ingredient compound, botanical mixture; pharmaceuticals vitamins, minerals, amino acid, enzymes, terpene, are then added. The mixture is liquid form.

In one embodiment delivery system further contains an effective amount of the following essential ingredients: sweeteners thickening/gelling agents to provide consistency and/or chewable texture to the delivery system in solid or liquid form.

In one embodiment the delivery further contains an effective amount of the following essential ingredients:/filtered water/juice/broth liquid infused thereof, mixed and or agitated with some not limited to agar; gelatin, pectin, vegetable, grain/vegetable starches, as thickeners.

As used herein the term “effective amount” is used to define the amount that will provide a beneficial effect or response in a patient. For example, a chemical response to mushroom mycelia compound, vitamin- and mineral-containing supplements varies from patient/condition(s) and symptoms presented, it should be understood that effective amounts of the vitamins and minerals will vary, respectively.

As used herein the effective pharmaceutical amount should be calculated determined through further professional laboratory testing and analysis.

The present invention may be used alone or in combination with one or more method, techniques, mechanical, chemical, and other modification, encapsulation, packaging, and the like for delaying the release of the nutrient, e.g., a capsule, a gelcap or even a coating. Examples of capsules include animal, vegetable, polymeric, mixtures, and combinations thereof. The coating (type, thickness, etc.) may be applied to a sufficient thickness such that part or the entire coating does not dissolve in the gastrointestinal fluids.

In one embodiment the delivery system further contains, nutritional yeast, malt extracts, dextrose. citric acid, ascorbic acid.

The present invention may be used alone or in combination with one or more method, techniques, mechanical, chemical, and other modification, creating an edible or suppository.

The embodiments herein relate to a delivery system and methods of production.

In one embodiment of the invention provides a solid delivery system comprising:
(I) 0.15-50 wt % active ingredient compound and botanical mixture
(II) one or more, thickening binding agents; stabilizers
(III) at least one fruit/vegetable juice and/or extract; and sweetener, enhancement, vitamins, mineral, amino acids, fruit vegetable herbal, essential oils/extracts derivatives of
   The described solid delivery system can be aliquoted into a troche, confection, or confection like, or chewable tablet, or gummy having a total weight of about 0.5-6 grams.

In one embodiment of the invention provides a liquid delivery system comprising:
(I) 0.15-50 wt % active ingredient compound and botanical mixture
(II) one or more, thickening binding agents; stabilizers
(III) at least one fruit/vegetable juice and/or extract; and sweetener, enhancement, vitamins, mineral, amino acids, fruit vegetable herbal, essential oils/extracts, or derivatives thereof.
   The liquid delivery system can be aliquoted into a container providing a single serving of 0.2 ml to 480 ml in volume.

In one embodiment, present invention provides a method of solid state myceliation of an agricultural substrate for producing functional composition by inoculating the delivery system directly, and or solid aqueous or liquid with pure strains of mushroom mycelial fungal culture selected from a group consisting from Sordariomycetes, Basidiomycota and Ascomycota. Species studied, noted yet not limited to Hericium erinaceus, Tremella fuciformis, Grifola frondosa, Agaricus blazei, Cordyceps sinensis, Cordyceps militaris, Flammulina velutipes, Ganoderma lucidum, Grifola frondosa, Letinula edodes, Phellenis linteus, Polyporus umbellatus, Inonotus obliquus, Pleurotus ostreatus, P. eryngii, P. citrinopileatus, P. djamor, Trametes versicolor, Volvariella volvacea, inonotus, Hypsizygus marmoreus, Pholiota nameko, Tuber melanosporum, Moschella hortensis, Bacopa Monnieri, Sarcodon spp., Antrodia camphorata, Pleurotus giganteus, Lignosus rhinocerotis, obliquus, Trametes versicolor, Poria cocos, and combinations not limited to and thereof;

The ingredients for the infused beverage can derived from juice, sap, extract, essential oil, dried, flower, seed, root, trim, leaf, bud; Cruciferious vegetation, fruit, botanicals, herbs, peels, skins, seeds, stems, leaves, or infusion and or derivatives can be of or contain ayurvedic plants, Aloe, Cacti, Cannabis, CBD, coconut, apples and pears, apricots, cherries, almonds, peaches, strawberries, raisins, coffee, lemons, oranges and grapefruit; tomatoes, potatoes, peppers, eggplant, African blue basil, Angelica, Anise (Pimpinella anisum), Aniseed myrtle (Syzygium anisatum), Annatto (Bixa orellana), Apple mint (Mentha suaveolens), Artemisia vulgaris, Mugwort, Asafoetida (Ferula assafoetida), Ajwain (Trachyspermum ammi), Allspice (Pimenta dioica), Amchur—mango powder (Mangifera), Angelica (Angelica archangelica), Anise (Pimpinella anisum), arrowroot, Arugula/Rocket, Berberis, Barbados Cherry, Banana, Barley, Basil (Ocimum basilicum), Bay leaves, Baobob, Bistort (Persicaria bistorta), Black cardamom, Black cumin, Blackcurrant, Black limes, Black Berry, Black Strap molasses, Bladder wrack (Fucus vesiculosus), Blue Cohosh, Blue-leaved Mallee (Eucalyptus polybractea), Bog Labrador Tea (Rhododendron groenlandicum), Boldo (Peumus boldus), Bolivian Coriander (Porophyllum ruderale), Borage (Borago officinalis), Berberis, Beet Root, Banana, Banana Peel, Basil (Ocimum basilicum), Bay leaves, Bistort (Persicaria bistorta”), Boldo (Peumus boldus), Borage (Borago officinalis), Butterfly Pea Flower, Brazilian Pepper, Black Pepper, Cacao, Cocoa, Cacti species, Calamus, Candlenut Calendula, Calumba (Jateorhiza calumba), Brambleberry (assorted beery), Chamomile, Cruciferous vegetation, Candied flowers, Caper (Capparis spinosa), Caraway, Cardamom, Carob Pod, Cassia, Casuarina, Catnip, Cat's Claw, Catsear, caspacian, Cayenne pepper, Celastrus Paniculatus, Comfrey, Celery, Celery seed, Centaury, Chervil (Anthriscus cerefolium), Chickweed, Chicory, Chile pepper, Chili powder, Cinchona, Chives (Allium schoenoprasum), Cicely (Myrrhis odorata), Cilantro (see Coriander) (Coriandrum sativum), Champagne Grapes, Cinnamon (and Cassia), and bark, Cinnamon Myrtle (Backhousia myrtifolia), Ceylon Cinnamon, cherry, Clary, Cleavers, Clover, Cloves, Coltsfoot, Comfrey, Common Rue, Condurango, Coptis, Coriander, Costmary (Tanacetum balsamita), Couchgrass, coffee, Cocoa, Cacao, cocoa butter, Cola Nitida acuminata, Cow Parsley (Anthriscus sylvestris), Cowslip, Cramp Bark (Viburnum opulus), Cress, Cuban Oregano (Plectranthus amboinicus), Cudweed, Cumin, Curry leaf (Murraya koenigii), Centaury, Chervil (Anthriscus cerefolium), Damiana (Turnera aphrodisiaca), Dandelion (Taraxacum officinale), Demulcent, Devil's claw (Harpagophytum procumbens), Dill seed, Dill (Anethum graveolens), Dorrigo Pepper (Tasmannia stipitata), Echinacea, Echinopanax Elatum, Edelweiss, Elderberry, Elderflower, Elecampane, Eleutherococcus senticosus, Epazote (Chenopodium ambrosioides), Ephedra, Eryngium foetidum, Eucalyptus, Fennel (Foeniculum vulgare), Fenugreek, Feverfew, Figwort, Five-spice powder (Chinese), Emmenagogue, Epazote (Chenopodium ambrosioides), Ephedra—Eryngium foetidum, Eucalyptus, Eyebright, Fennel (Foeniculum vulgare), Fenugreek, Feverfew, Figwort, File powder, Fo-ti-tieng, Fumitory, Galangal, Garam masala, Garden cress, Garlic chives, Garlic Geranium, Ginger, Gideon, Ginkgo biloba, Ginseng, Ginseng, Siberian (Eleutherococcus senticosus), Gentian, Goat's Rue (Galega officinalis), Goada masala, Golden Rod, Golden Seal, Gotu Kola, Grains of paradise, Grape seed extract, Green tea, Ground Ivy, Guaco, Gypsywort, Hemp, Hemp Marijuana Hybrid Hawthorn (Crataegus sanguinea), Hawthorne Tree, Hemp, Herbes de Provence, Hibiscus, Holly, Holy Thistle, Hops, Horehound, Horseradish, Horsetail (Equisetum telmateia), Hibiscus, Hyssop (Hyssopus officinalis) Jalap, Jack Fruit, Jasmine, Jiaogulan (Gynostemma pentaphyllum), John the Conqueror, Juniper, Key lime, Kaffir Lime Leaves (Citrus hystrix, C. papedia), Kaala masala, Knotweed, Kokam, Labrador tea, Lady's Bedstraw, Lady's Mantle, Land cress, Lavender (Lavandula spp.), Ledum, lemon, Lemon Balm (Melissa Officinalis), Lemon basil, Lemongrass (Cymbopogon citratus, C. flexuosus, and other species), Lemon Ironbark (Eucalyptus staigeriana), Lemon mint, Lemon Myrtle (Backhousia citriodora) Lemon Thyme, Lemon verbena (Lippia citriodora), Lychee, Licorice—adaptogen, Lemon, Lime, Lime Flower, Limnophila aromatica, Lingzhi, Lingonberry, Linseed, Liquorice, Long pepper, Lovage (Levisticum officinale), Luohanguo, magic berry, Mace, Magnolia (flower, bark, wood), Mahlab, Malabathrum, Manchurian, Melon, Thorn Tree (Aralia manchurica), Mandrake, Mango, Marijuana, Marjoram (Origanum majorana) Marrubium vulgare, Marsh Labrador Tea, Magnolia, Marshmallow, Mastic, Meadowsweet, Mei Yen, Mint (Mentha spp.), Milk thistle (Silybum), Bergamot (Monarda didyma), Motherwort, Mountain Skullcap, Mullien (Verbascum thapsus), Mustard, Mustard seed, Nashia inaguensis, Neem, Nepeta, Neroli, Nettle, Nigella sativa, Nigella (Kolanji, Black caraway), Noni, Nutmeg (and Mace), Oenothera (Oenothera biennis et al), Olida (Eucalyptus olida), Oak, Oregano (Origanum vulgare, O. heracleoticum, and other species), Orris root, Osmorhiza, Olive Leaf (used in tea and as herbal supplement), Orange Bitter Orange/Sour Orange, Pea flower Clitoria ternatea, Palo Azul, Panax quinquefolius, Pandan leaf, Paprika, Parsley (Petroselinum crispum), Passion Flower, Patchouli, Pea Flower, Pennyroyal, Pepper (black, white, and green), Pear, Peach, Peppermint Gum (Eucalyptus dives), Perilla, Plantain, Pineapple, Pomegranate, Ponch phoran, Poppy seed Primrose (Primula) candied flowers, tea, Psyllium, Purslane, Quassia, Quatre épices, Ramsons Ras el-hanout, Raspberry (leaves), Reishi, Restharrow, Rudarelis, Rhodiola Rosea, Riberry (Syzygium luehmannii) Roman chamomile, Rooibos, Rosehips, Rosemary (Rosmarinus officinalis) Rowan Berries, Rue, Safflower, Saffron, Sage (Salvia officinalis), Saigon Cinnamon, St John's Wort, Salad Burnet (Sanguisorba minor or Poterium sanguisorba), Salvia, Sichuan Pepper (Sansho), Sassafras, Savory (Satureja hortensis, S. montana), Schisandra (Schisandra chinensis), Scutellaria costaricana, Senna (herb), Sea moss, Senna obtusifolia, Sesame seed, Sheep Sorrel, Shepherd's Purse, Sialagogue, Siberian Chaga, Siberian ginseng (Eleutherococcus senticosus), Siraitia grosvenorii (luohanguo), Skullcap, Sourgum, Sloe Berries, Sonchus, Sorrel (Rumex spp.), Southernwood, Spearmint, Speedwell, Spilanthes Toothache Plant, Squill, Succulents, Star fruit, Star anise, Stevia, Strawberry, Strawberry Leaves, Suma (Pfaffia paniculata), Sumac, Summer savory, Sutherlandia frutescens, Sweet grass, Sweet cicely (Myrrhis odorata), Sweet woodruff, Sweet Almond, Bitter Almond, Szechuan pepper (Xanthoxylum piperitum), Tacamahac Tamarind, Tansy, Tarragon (Artemisia dracunculus), Tea, Teucrium polium, Thai basil, Thistle, Thyme, Tonka Bean, Toor Dall, Tormentil, Tribulus terrestris, Tulsi (Ocimum tenuiflorum), Turmeric, Uva Ursi also known as Bearberry, Vanilla bean (Vanilla planifolia), Vasaka, Vervain, Vetiver, Vietnamese Coriander, Wasabi, Watercress, Watermelon seed, watermelon, Wattleseed, Wild ginger, Wild Lettuce, Wild thyme, Winter savory, Witch Hazel, Wolfberry, Wood Avens, Wood Betony, Woodruff, Wormwood, Yarrow, Yerba Buena, Yerba Mate, Yohimbe, Za'atar, Zedoary Root.

Cordyceps sinesis and militaris (The Zombie Corpse Mushroom). The remains of farmed insects are inoculated by this amazing fungus and are then hand-collected, dried and have been used in Traditional Chinese Medicine for centuries once prized by emperors to treat fatigue, sickness, and low sex drive. Cordyceps contains adenosine and can stimulate the production of ATP—one of the main sources of energy in your body's cells to improve stamina in athletic performance. The remains of the insect and fungi have been hand-collected, dried, and used in Traditional Chinese Medicine for centuries to treat fatigue, sickness, kidney disease and low sex drive. Supplements and products containing Cordyceps extract have become increasingly popular due to their many purported health benefits.

Cordyceps sinensis, also called DongChongXiaCao (winter worm, summer grass) in Chinese, is becoming increasingly popular and important in the public and scientific communities. This study summarizes the chemical constituents and their corresponding pharmacological actions of Cordyceps sinensis. Many bioactive components of Cordyceps sinensis have been extracted including nucleoside, polysaccharide, sterol, protein, amino acid, and polypeptide. In addition, these constituents' corresponding pharmacological actions were also shown in the study such as anti-inflammatory, antioxidant, antitumour, antiapoptosis, and immunomodulatory actions. Therefore, can use different effects of C. sinensis against different diseases and provide reference for the study of Cordyceps sinensis in the future. The genus Cordyceps is an important kind of medicinal fungi belonging to the Ascomycota, Pyrenomycetes, Hypocreales, and Clavicipitaceae [1-5]. Cordyceps are specific macrofungi because of their characteristic parasitic habit on larvae and pupae of insects. As a pleomorphic fungus distributed worldwide, Cordyceps is particularly abundant in tropical forests and humid temperate [3-5]. Within the genus Cordyceps, over 400 species have been described so far [4, 5], of which Cordyceps sinensis, also called as “winter worm, summer grass,” is recognized as the most famous tonic herb in traditional Chinese medicine (TCM) for centuries.

Lion's mane mushrooms (Hericium erinaceus) are white, globe-shaped fungi that have long, shaggy spines and offer a range of health benefits, including reduced inflammation and improved cognitive and heart health Inflammation and oxidation Antioxidants may fight both inflammation and oxidation in the body Inflammation contributes to many medical conditions, including diabetes, heart disease, and autoimmune diseases. A 2012 study evaluating the medicinal potential of 14 types of mushroom found that lion's mane had the fourth highest antioxidant activity, which researchers described as “moderate to high”. Immune function that lion's mane mushrooms may boost the activity of the intestinal immune system. Anxiety and depression modulating extracts from lion's mane mushrooms may be beneficial in the treatment of anxiety and depression. In a 2015 study, mice that consumed lion's mane mushroom extract displayed fewer depressive behaviors and had blood markers that indicated lower depression. The researchers suggest that this is due to the extract's anti-inflammatory effects that are useful for treating depressive disorders, including cognitive function, menopausal symptoms and poor sleep quality, researchers have concluded that the mushrooms may have the potential to treat or prevent diseases that cause a decline in cognitive health, such as Parkinson's disease and Alzheimer's disease. One of the complications of diabetes is nerve damage resulting from prolonged periods of high blood sugar. A 2015 study on rats, in which they ingested lion's mane extract for 6 weeks, showed positive results, including lower blood sugar levels, reduced feelings of nerve pain, and improved antioxidant activity.

Lion's mane may help digestive health by fighting inflammation, which could be beneficial for people with inflammatory bowel disease (IBD). The mushroom may also boost immune function and encourage the growth of good bacteria in the gut may protect against stomach ulcers, provide healing benefits for skin wounds. A study on rats with neck wounds found that topical application of lion's mane extract led to faster healing. extracts of lion's mane mushrooms may encourage nerve cells to grow and repair more quickly. One study showed that rats with nerve damage receiving a daily extract of lion's mane mushrooms had quicker nerve regeneration.

Jack Herrer Cannabis strain containing Linalool (0-0.05%) Pinene (0-1.0%) Limonene (0-0.9%) Caryophyllene (0.01-0.4%) Myrcene (0.1-0.3%) for example Terpenes that are a class of organic hydrocarbons, lactones, alcohols, acids, aldehydes, and ketones with strong aromatic properties and are primary constituents containing an impressive array of biological activities. Jack Herer is a sativa-dominant cannabis strain that has gained as much renown as its namesake, the marijuana activist and author of The Emperor Wears No Clothes. Combining a Haze hybrid with a Northern Lights #5 and Shiva Skunk cross, Sensi Seeds created Jack Herer hoping to capture both the cerebral elevation associated with sativas and the heavy resin production of indicas. Its rich genetic background gives rise to several different variations of Jack Herer, each phenotype bearing its own unique features and effects. However, consumers typically describe this 55% sativa hybrid as blissful, clear-headed, and creative while addressing musculoskeletal and Neuropathic pain, Anxiety, depression, migraines, headache, and general stress.

Cacti with a wide range of health benefits. These include their ability to aid in weight loss, cancer prevention, improving skin health, and protecting heart health. These cacti also help regulate and improve digestion, boost the immune system, optimize metabolic activity, build strong bones, cure insomnia, and reduce inflammation throughout the body.

For example, the nopales have been used in traditional medicine for disease treatment and prevention. In proven studies cactus plants have been used to treat: neurological disorders, migraines, glaucoma, wounds, fatigue, liver conditions, and ulcers lowering blood sugar, healing wounds, and lowering cholesterol. studies have confirmed antioxidant and anti-inflammatory properties.

The bark of Cinchona is an essential medicine and component in bitters and Tonics is used for digestive issues stomach upset, bloating, Blood Vessel disorders including hemorrhoids, and Varicose veins. Cinchona also contains quinine which is a powerful antispasmodic. Citrus provides Vitamin C. This family of fruits offers antioxidants folic acid, boosting reproductive system, sperm count and improving fertility. Damiana is said to help enhance sexual health and treat a vast array of symptoms from diabetes to anxiety. Prized for centuries by indigenous cultures as an aphrodisiac and bladder tonic.

Saw palmetto Honey's impressive benefits include its ability to improve hair growth, reduce prostate enlargement, treat urinary tract conditions, stimulate sexual activity, reduce impotence, improve the health of the kidneys, boost muscle mass, increase nutrient uptake efficiency, and even improve the immune system. Banana is rich in magnesium which is nature's ‘chill pill’. Oak with its deep flavor profile is rich with tannins as well as a powerhouse anti-inflammatory. Maca is a powerful mood enhancer that boosts sports performance and energy, increases libido, improves male fertility, and relieves symptoms of menopause.

Spilanthes, Electric Daisy, Buzz Buttons, Toothache Plant may have many names, but it is also known as a powerful aphrodisiac. Butterfly Pea flowers have a nootropic effect and trigger vivid dreams, enhanced memory, and brain function. This blue flower from Southeast Asia produces a brilliant blue liquid, that, when mixed with the acidity of lemon, changes the hue to a pink/purple tone. It has strong anti-aging properties and helps maintain healthy blood sugar levels. It also promotes eye health, UTI protection destroys free radicals. A natural fever reducer and reduces factors of heart disease. Jackfruit is rich in anti-oxidants. This, along with its antioxidant and phytonutrient properties is high Vitamin C. Tulsi Holy Basil, Sacred Basil tackles stress, anxiety, and inflammation. It creates a general sense of well-being, working to boost energy and enhance focus in order to deal with ongoing conflict and stress. As an adaptogen with anti-inflammatory and antioxidant properties, holy basil provides all of these benefits. It can even help people with arthritis and fibromyalgia.

Lemon verbena relieves anxiety and depression. Its sedative and aphrodisiac properties make it helpful for relaxing the mind and uplifting and improving one's mood. Fascinating health benefits are its ability to protect muscles, reduce inflammation, boost the immune system, calm the stomach, reduce fevers, soothe nerves, and clear up congestion. It is also popularly used for weight loss.

Honey offers anti allergen, antibacterial activity and immunomodulatory property, beneficial enzymes medical care and within agriculture promote root growth in plants and provide microbial nutrients Saw palmetto honey has been reported to exhibit antimicrobial activity against pathogenic bacteria such as Staphylococcus aureus (S. aureus) and Helicobacter pylori (H. pylori) content in enzymes, antioxidants, vitamins, acids, fitonutrients and a cancer fighter.

Water is a basic ingredient in confections and soft drinks since it is the vehicle or liquid portion in which the remaining ingredients are dissolved or dispersed. Purified water, distilled, and spring water were used in the creation beverage line contained in of the present invention. Water of a standard quality is required in order not to adversely affect beverage taste, odor, or appearance. The water must be clear, colorless, free from objectionable minerals, tastes, and odors, free from organic matter, low in alkalinity and of acceptable microbiological quality based on industry and government standards. Water is present in the beverage at a level of from about 80% to about 99.9% by weight of the beverage.

Various sweeteners are suitable for use in the present invention if insufficient sweetness is derived from the natural sugars present. Sugar cane, honey, agave, caster sugar, fruit derived sugars, natural crystalline or liquid sweeteners such as sucrose, fructose, glucose, levulose, high fructose corn syrup, invert sugar and others can be used. To achieve the desired beverage uniformity, texture, and taste, standardized liquid sugars as are commonly employed in the beverage industry can be used. These are free of traces of nonsugar solids which could adversely affect the flavor, color, or consistency of the beverage. Artificial or non-nutritive sweeteners are also suitable for use in the present invention. Such sweeteners include saccharin, cyclamate, aspartame, other dipeptides, acesulfame potassium, and others. Natural sweeteners are present in an amount of from about 0.1% to about 25% by weight of the beverage, preferably from about 10% to about 14% by weight. Non-nutritive sweeteners are employed at a level of milligrams per fluid ounce according to their sweetening power and the regulatory provisions of the country where the beverage is to be marketed. Sweeteners also affect the body or texture of the beverage.

Sugars syrups and liquids can be infused fermented stored aged in vessels, for example as liquor is aged in oak barrels to create complexity in flavor or promote beneficial bacteria growth. Sugars syrups and liquids can be infused fermented stored and or aged in vessels to create complexity in flavor or promote beneficial bacteria growth.

Acid is used for several functions. These include lending tartness to taste, enhancing palatability, increasing thirst quenching effect, modifying sweetness, and acting as a mild preservative. Citric acid and malic acid are most commonly used in confections while phosphoric acid is most commonly used in cola beverages. Other acids in addition to phosphoric acid suitable for use in the beverage of the present invention include citric, malic, tartaric, lactic, formic, ascorbic, hydrochloric, sulfuric, fumaric, and adipic. Preferred for use herein is a phosphoric acid. The acid is used in solution form in an amount of from about 0.01% to about 0.5% by weight of the beverage, preferably from about 0.05% to about 0.25% by weight of the beverage, depending upon the acid used. Benzoic acid, sodium benzoate, or other suitable compounds, can be added as a preservative. The level of preservative used is adjusted according to the planned final product pH, as well as an evaluation of the microbiological spoilage potential of the particular beverage medium. The correct amount of acid enhances beverage flavor. Too much acid destroys flavor and results in sourness.

If required coloring and flavoring may be in form of an extract, essential oil liquid and or powder can be provided by functional ingredients, outsourced or store bought. The flavoring is preferably in the form of an extract and can be prepared by a cold hydroalcoholic extraction process. This process produces essential oil flavor extracts of high clarity and significant organic superiority. This process for producing a flavor extract from essential oil in example of steps of (a) combining an essential oil or mixture thereof with an extraction solvent comprising from about 45% to about 75% ethanol, from 0% to about 5% of a food-compatible glycol, and from about 25% to about 55% water; (b) storing the mixture at a temperature of at most 45° F. (7° C.) for a period of at least 72 hours, to provide an aqueous flavor extract phase and a terpene phase having a lower density than the aqueous flavor extract phase; and (c) separating the terpene phase from the aqueous flavor phase.

Process times can range from about one hour to about two weeks. However, it has been determined that the extraction process itself is about 90% complete in three days at the temperatures specified herein. Thus, process times greater than about three days are effective primarily to allow more complete separation of the terpene phase from the aqueous phase, providing a “cleaner” cut and to aid in minimizing the need for subsequent filtration. High agitation promotes the extraction part of the process by increasing the terpene phase/aqueous phase interfacial area, but high agitation does not permit the extraction system to separate. Accordingly, the extraction system is permitted to rest after initial agitation to provide a clean separation. Following the resting period, simple physical separation is all that is required. The separation process can take advantage of the fact that the aqueous phase has a higher density than the terpene phase. The resulting product is the clear flavor extract used in this invention. If desired, the extract can also be filtered through paper, using diatomaceous earth, or activated charcoal as a filtration aid.

The flavoring can also be used in the form of an emulsion. The emulsion is prepared first by mixing the flavorings together, and then combining the mixture with an emulsifying agent. Flavorings also include juice concentrates as previously defined. An emulsifier which is a water-soluble material such as gum acacia, modified starch, carboxymethylcellulose, gum tragacanth, gum ghatti or other suitable gums is used as the emulsifying agent. The emulsifier comprises from about 5% to about 30% of the mixture of flavorings and emulsifier. Other suitable ingredients, such as acid or a preservative, can be incorporated into the emulsion if desired.

It can be appreciated that other specific embodiments of this invention can be devised without departing from the scope and spirit of the invention, and without losing its advantages. Flavoring as described is to be understood as being nonspecific to a delivery system. In particular, liquid mixtures combining flavoring emulsions, juices, or other means of adding flavors to beverages can be prepared for use in a solid delivery system. This object is achieved when liquid mixtures containing flavoring are solidified into a gel-like consistency through the use of a thickening agent.

Carbon dioxide and or carbonation by natural fermentation may be used to provide dimension and effervescence to the beverage of the present invention. Any of the techniques and carbonating equipment known in the art for carbonating beverages can be employed. Carbon dioxide enhances beverage taste and appearance and aids in safeguarding beverage purity by inhibiting and destroying objectionable bacteria. The beverage of the present invention has a carbon dioxide level of from about 0.5 to about 6.0 volumes carbon dioxide. One volume of carbon dioxide is defined as the amount of carbon dioxide absorbed by any given quantity of water at 60° F. (16° C.) temperature and atmospheric pressure. A volume of gas occupies the same space as does the water by which it is absorbed. Since there is a definite relationship between taste and carbonation, it is important to maintain the carbonation within the desired range. Preferred for use herein is natural carbonation.

Natural Fermentation techniques can be used for carbonation, flavor, probiotic benefits. In certain circumstance Caffeine can be added to the beverage of the present invention if desired. The amount of caffeine added is determined by the regulatory provisions of the country where the beverage is to be marketed, and typically is at a level of 0.02 percent or less by weight of the beverage. The caffeine must be of a purity acceptable for use in foods and beverages.

It can be further appreciated that other specific embodiments of this invention can be devised without departing from the scope and spirit of the invention, and without losing its advantages. Minor ingredients can be reasonably altered or eliminated without departing from the scope of this invention. In particular, carbonated juice-based beverages having the taste of a popular soft drink, energy drink or alcoholic beverage and can be prepared in a liquid or powdered form. This object is inventively achieved in that the beverage contains additionally juices botanicals and mushroom mycelium and may contain Cannabis, CBD products.

The combination of juice as a thirst quencher and additional admixed ingredients mushrooms and or mycelium in combination or without cannabis, CBD, hemp; to provide effects and are active ingredients infused into a beverage. For example, juices may be employed so that the flavor of the beverage can be typical of a ginger beverage in general the beverage comprises water, fruit, berry, botanicals or citrus juices, acid, and natural flavorings. Juices may also be employed, so that the flavor can be typical of a tropical flavored beverage in general the beverage comprises water, fruit, berry, botanicals or citrus juices, acid, and natural flavorings.

Juices can further be employed, so that the flavor of the beverage can be typical of brambleberry, a berry flavored beverage. In general the beverage comprises water, fruit, botanicals or citrus juices, acid, and natural flavorings. Juices may additionally be employed so that the flavor of the beverage can be typical of a jackfruit flavored beverage. In general, the beverage comprises water, botanicals, fruit, or citrus juices, acid, and natural flavorings. In one embodiment, an infused confection of a mixture of Brambleberry or a singular or plural species of Wild berries is used.

Juices, however, may not always be desired in the product. For example, the flavor of the beverage can be typical of a Chocolate flavored beverage in general the beverage comprises water, and or type of milk where certain flavorings of fruit, botanicals, berry or citrus juices, acid, and natural flavorings would respectively change. A sweetener can be added if there is insufficient sweetness from the juices. Carbonation in the form of carbon dioxide is added for effervescence. A preservative and binding agent can be added if necessary. Vitamins, supplements, amino acids, probiotics, and prebiotics can be added as an optional ingredient. To create a sip-ability a bitter component may be added. Further addition can and may containing fruit, berry, or citrus juice. Natural colors derived from the ingredients present. One or more juices, extracts botanicals, floral extract, alone or mixed with other suitable, is employed to provide the effects and enjoyability to a beverage without the use of harmful ingredients. The beverage may comprise carbonated water, juices, acid, and natural flavorings. No artificial flavorings are employed.

Juices suitable for use in the present invention include citrus, fruit, or berry juices. Juices can be employed in the present invention in the form of a concentrate, puree, single-strength juice, or other suitable forms. The term “juice” as used herein denotes single-strength fruit, berry, or citrus juices, as well as concentrates, purees, milks, and other equivalent forms not to exclude artificial embodiments.

In one embodiment Components such as calcium, folic acid, vitamin A, vitamin B1, vitamin C and vitamin E are contained in juice. The ingredients of energy drink taurine, caffeine, glucuronolactone, vitamin B2, vitamin B6 and vitamin B12 may be included other health-promoting or energizing ingredients such as magnesium, or dextrose are possible.

The infused beverage can be of any standard drink readily on the market or specially formulated per circumstance. The formulation, composition, or substance of this invention may be prepared in a number of ways, including as a soft drink, powdered mix, as a juice, as a tonic, tincture, liquor, liqueur, energy drink, or in a solid delivery system as a gummy, confection, troche, chewable tablet, or as a food additive prepared for recreational supplementation or pharmaceutical use.

The infused beverage should be all organic property with carefully selected ingredients based on circumstance and or condition requirements and or demands; be of any standard drink readily on the market or specially formulated. The formulation, composition, or substance of this invention may be prepared in a number of ways, including as a soft drink, powdered mix, as a juice, as a tonic, tincture, liquor, liqueur, energy drink, or in a solid delivery system as a gummy, confection, troche, chewable tablet, or as a food additive prepared for recreational supplementation or pharmaceutical use.

Similarly, an infused solid delivery system can be of any confectionary product readily on the market or specially formulated per circumstance The formulation, composition, or substance of this invention may be prepared in a number of ways, including as a gummy, powdered mix, tablet, troche, lozenge, gummy, confection, or chocolate as a food additive prepared for recreational supplementation and pharmaceutical use.

Alternatively, the formulation may be any formulation using any combination of mushroom and or mycelia CBD cannabis, hemp prepared in any manner and or combination will be within the spirit of the invention.

Several readily available store-bought products on the market were included in this study. Alternating mushroom compounds, extracts and dried mycelium and fruiting bodies were tested alone then combined with CBD prepared extract, activated hash oil and plant matter were infused and combined in different method all tested in varying strengths with great success. dosages would be adjusted based on market and effects desired in embodiments.

### Example 1

Readily available energy drink on the market combined 0.1 wt. % to 0.6 wt. %, 0.4 wt. % in active ingredient. It may be preferred the invention of an energy beverage can have a caffeine content of 0.01 wt. % to 0.05 wt. %, preferably 0.032.% By weight. In addition, it can be provided that the energy drink comprises calcium and/or potassium, of 0.03.% To 0.2 wt. %, preferably 0.12.% calcium or potassium. As such energy drinks enjoy popularity, the beverage can meet the increased need for calcium by the user compared to the levels of calcium currently available in fruit juices, vitamins, energy drink for further food supplement vitamin A, vitamin B1, vitamin B2, vitamin B6, vitamin B12, vitamin C, vitamin E and/or folic acid.

An example of an energy beverage or product for supplementation may comprise 0.00001% wt % to 0.001 wt. Vitamin A, preferably 0.0008 wt. %; 0.00005 wt. % to 0.005 wt. % of vitamin B1, in particular 0.0024 wt. %; 0.0001 to 0.001 wt. % of vitamin B2, preferably 0.0006 wt. %; 0.001 wt. % to 0.005 wt. % of vitamin B6, preferably 0.002 wt. % by weight; 0.000001.% to 0.000005 wt. % vitamin B12, in particular 0.000002 wt. % to 0.000003 wt. %; Vitamin B12, 0.02 wt. % To 0.2 wt. %; Vitamin C, in particular 0.18 wt. %, 0.0001 wt. % to 0.02 wt. %; vitamin E, in particular 0.01 wt. %; and 0.000001 wt. % to 0.0006 wt. % folic acid, preferably 0.000004 wt. %. Mushroom mycelia, extracts, fruiting bodies would be added to provide beneficial properties as described herein.

### Example 2

One particular botanical variation chosen for medical property or flavor according to the subject invention is termed “Purple Empress”, based on its purple color, signifying power and royalty—reminiscent of the tropics with a grape note, hints of basil, and lemon. Ingredients were specifically paired to excite the taste buds, being fruity and fun. Not just a mood lifter, the Purple Empress flavor provides energy stamina and vitality. Ingredients: Butterfly Pea Flower, Jackfruit, Tulsi Holy Basil, Sacred Basil, Lemon Verbena, Dried Cordyceps Mycelium, Purple Mystic mycelium. Due to availability of ingredients, certain products were used these are just a mere suggestion of Mushroom strain, Mycelium strain, Cannabis, CBD THC, Terpenes combinations can vary based on ingredient profile condition to be addressed and or effect desired. Cordyceps and Purple Mystic strains were both utilized in separate studies. Mushroom mycelium was obtained through sterile procedure in a home lab. The liquid cultures of mycelium were derived from the fruiting bodies of Cordyceps militaris in sterile jars with a liquid solution of distilled water and saw palm honey and a nutritive and antibacterial. The mycelium disk was removed, and dried, ground, and pulverized Purple Mystic fruiting bodies were prepared into an extract by double water extraction. Mushroom mycelium and or fruiting bodies may be utilized in dried, liquid state, outsourced and or locally farmed. Binding agents such as soy lecithin, xanthan gum, and gum arabic can be used. Gum arabic is a preferred binding agent, due to taste and clarity of the final product. with the beverage can comprise mushroom extract Koa Samui alone, or can further comprise 35 mg activated Jack Herer Cannabis extract, or can further comprise Cordyceps mushroom mycelia. This results in a liqueur with approximately 35 grams of THC combined with 0.75 grams of psilocybin mushroom to create a low to medium light psychotropic effect. An expert flavor panel evaluated the beverage determined it tasted like sweet, tropical, grape-like juice. Effects were felt within 20 minutes in one of the panelists, effects were felt within 45 minutes in two of the panelist's effects lasted between 3 and 5 hours. These results will vary based on product, prior usage, tolerance, metabolic rate of individual.

To create a product profile and shelf stability, Caster sugar was used as a sweetening agent. Sorbitol was used as a preservative. Gum Arabic was utilized for its flavor and binding capabilities. These are preferred, but other ingredients with similar properties can be use. For example, other preservatives commonly used in soft drinks are sulfur dioxide, sorbic acid and its salts and benzoic acid and its salts. Sulphur dioxide and sulphites have been extensively used as antimicrobials for many centuries and are highly effective. Simple syrup and water extraction methods were utilized. Local foraged, as well as locally farmed ingredients combined with outsourced essential oils and extracts were utilized to create flavor profiles and handpicked to create a symbiotic relationship among ingredients. Carbon dioxide gas can be used to provide carbonation to the final beverage product. Ingredient extracts may be used as a tincture in condensed form or further processed into a shot like or full-size carbonated beverage. Alternative ingredients and amounts and or methods of should be introduced when preparing commercial quantities.

### Example 3—Electric Aphrodesia “the Electric Teaser”

Complex flavors of citrus, honey and charred oak with a floral finish were used to stimulate the mood and soul resembling a fine liqueur that can be sipped or further mixed into a beverage. Ingredients include Honeybell Orange, Pink Grapefruit, Damiana, Saw Palmetto Honey, Saffron, Fenugreek, magnolia bark Rose, Hibiscus, Oak, Maca, Banana Peel Tea, Lions mane, Myrcene terpene extract, Koa Samui mushroom mycelium. The beverage is preferably imbibed using a drinking vessel rimmed with Electric Daisy-Spilanthes and honey to further entice and electrify the senses. Terpenes can be included to create an entourage effect and add another level of flavor. Terpene profiles vary upon strain and effect desired. Linanool, Alpha Pinene, Beta Caryophyllene were all tested with great success. Beverage syrups can be prepared with or without Cannabis. To prepare this embodiment of the beverage, Saw Palmetto honey, wood, seeds, bark, botanicals were placed in a sanitized mason jar, and placed into a 110-degree water bath for 12 days. The low heat creates a decarboxylation process required to activate THC, Tea beverages were made from Floral components damiana 1 part botanical to 3 parts water can be added to the above beverage and the mixture placed in a 110-degree water bath for an additional 12 hours. The contents were strained to remove the solid components. Banana peel tea, magnolia and floral teas were combined. The Electric Aphrodesia could be sold as an energy shot, drink additive/mixer, soft drink, liquor. Citrus juices based on the flavor aphrodisiac property and in combination enhance the effects of psilocybin. Citric acid terpenes can be included. Binding agents such as soy lecithin, xanthan gum, and gum arabic can be used. Gum arabic is a preferred binding agent, due to taste and clarity of the final product. Mushroom psilocybin was obtained from Koa Samui mycellum and a double water extraction of Lions mane fruiting body. A first embodiment was prepared using ingredients with Koa Samui Mushroom Mycelium. A second embodiment was prepared wherein the beverage was infused with 35 mg activated Jack Herer cannabis. A third embodiment was prepared using Lions Mane extract 35 mg with activated Jack Herer cannabis. This has created a liqueur with approximately 35 grams of THC combined with 0.75 grams of mushroom. to create a low to medium light psychotropic effect. An expert flavor panel evaluated the beverage determined it tasted like herbal, floral, woodsy sweet with a sip-ability of an alcohol related beverage. Effects were felt within 20 minutes in 3 of the panelists; effects were felt within 45 minutes in two of the panelists; effects lasted between 3 and 5 hours. These results will vary based on product, prior usage, tolerance, metabolic rate of individual.

Sorbitol can be used as a preservative. Citric acid can be alternatively be used as a preservative, and is preferred for enhancing the citrus flavor. Gum Arabic was utilized for its flavor and binding capabilities. other commonly used ingredients in soft drinks can also be used and include sulfur dioxide, sorbic acid and its salts and benzoic acid and its salts. Caster sugar was used as a sweetening agent. Simple syrup and water extraction methods were utilized. Local foraged, as well as farmed ingredients supporting the local farming community, combined with outsourced essential oils and extracts were utilized to create flavor profiles and handpicked to create a symbiotic relationship of ingredients. Carbon dioxide can be included for carbonation of final product would add a further dimension to the beverage. Ingredient, including extracts, may be as a tincture in condensed form and aliquoted into a shot like or full-size carbonated beverage.

The infused beverage is created to treat seizures, said method comprising: (i) identifying a patient with Seizures; and (ii) administering an amount of the of the present invention to a patient; wherein said patient experiences reduced number of seizures due to administration, with reduced side effects, and a pleasing experience. In this circumstance Marijuana and other cannabis products with high content in Δ(9)-tetrahydrocannabinol (THC), utilized primarily for recreational purposes, are generally unsuitable for this indication, primarily because THC is associated with many undesired effects. Compared with THC, cannabidiol (CBD) shows a better defined anticonvulsant profile in animal models and is preferred for children. psilocybin in circumstance can trigger seizure activity until further study hallucinogens are not currently recommended, Lingzi, Reishi or Lions mane Mushroom would be used in combination with a high CBD formulation. THC would be included based on patient/consumer preference.

In one embodiment the present invention treats Arthritis, said method comprising: (i) identifying a patient with Arthritis; and (ii) administering an amount of the present invention to a patient; wherein said patient experiences joint pain relief due to said administration, with reduced side effects and a pleasing experience. Since 2001, medical cannabis (also known as marijuana) has been a legal treatment option in Canada for certain health conditions, including arthritis. An estimated two thirds of Canadians who use cannabis for medical purposes do so to help manage arthritis symptoms. A research team led by Christian Hertweck at the Leibniz Institute for Natural Products Research and Infection Biology, Jena, Germany, have identified compounds in mushrooms which they say are capable of treating rheumatoid and gouty arthritis. Nonsteroidal anti-inflammatory drugs called NSAIDs help relieve joint swelling, stiffness, and pain—and are among the most commonly used painkillers for people with any type of arthritis. The inflammation of joints and tissues in these common diseases are controlled by enzymes in the body. The researchers claim the compounds they have identified can block the action of the specific enzymes which cause the inflammation.

### Example 4

A confection was manufactured comprising mushroom extract (Coryceps militaris alone), and at least one specific botanical, e.g., elderberry or mullein, selected based on the symptoms or ailment for which relief was desired. For example, in circumstances where relief of respiratory conditions, symptoms, ailments, or disease, elderberry can be included in the composition for its immunological or antiviral properties. In the alternative, or in addition, mullein can be included in the composition for its muscle relaxing properties. Other suitable herbs or tinctures can also be used.

Europeanelder (Sambucus nigra), also known as black Elderberry is known to be beneficial for the prevention and treatment of influenza and upper respiratory infections, though there is still debate as to which stage of infection it is most effective in and even what the exact mechanisms of action are that lend elderberry its anti-viral effects. American elder (Sambucus canadensis) is a fast-growing, deciduous North American shrub that can reach up to 12 feet with flowers and berries similar to S. nigra. Elder flower extracts are also used to treat the common cold and influenza. The mechanism of action of elderberry and its primary anthocyanin compound, cyanidin 3-glucoside, affect the infectivity of the influenza virus. When evaluating pasteurized, whole-elderberry extract at different concentrations, as well as the isolated bioactive compound cyanidin 3-glucoside at different concentrations, it was found that these extracts had an inhibitory effect on influenza with higher concentrations having a greater effect on influenza viral activity at different stages of infection.

Mullein has been used for thousands of years to treat respiratory conditions. It is especially effective at relieving asthma, which causes airways to swell and results in symptoms like coughing, wheezing, and shortness of breath. Animal and human research suggest that mullein reduces inflammation thereby helping relax the muscles in your respiratory tract Both flowers and leaves of the plant are also used to treat other respiratory ailments, such as tuberculosis, bronchitis, tonsillitis, and pneumonia.

Based on the availability of ingredients, products were used including but not limited to Mushroom strains, Mycellium strains, Botanicals, or Terpenes combinations which can vary based on the ingredient profile condition to be addressed and or effect desired. Cordyceps strains were also utilized. Mushroom mycellium were obtained through sterile procedure in a home lab. The liquid cultures of mycellium were derived from the fruiting bodies of Cordyceps militaris in sterile jars with a liquid solution of distilled water and saw palm honey and a nutritive and antibacterial. The mycellium disk was removed and dried, ground and pulverized fruiting bodies were prepared into an extract by double water extraction. Mushroom mycellium and or fruiting bodies were utilized in dried or liquid state, outsourced, or locally farmed. An expert flavor panel evaluated the confections and determined it tasted like sweet, berry-like juicy. These are preferred, but other ingredients with similar properties can be used.

In one embodiment, the composition as described in this example is produced as a sweet pectin-based chewy textured confection as a delivery system for the beneficial ingredients. It is preferred to administer the confection to the patient before exposure to viral agents, or other infectious agents or allergens, as a preventive, but administration can continue while the patient suffers symptoms.

### Example 5

A solid form product, e.g., troche, lozenge, tablet, chewable tablet, confection, infused confection, or gummy product can be produced using the liquid compositions as prepared and described in Examples 1-3 as a starting material. These liquid starting materials can be solidified using any conventional means or process known or available in the art, including but not limited to, adding a thickener, solidifying agent, or gelling agent to the liquid starting material, or by drying the liquid starting material or by reducing the liquid starting material by heat to drive off water, aqueous liquid, or other volatile liquid to produce the solid form. Other processing steps, such as molding, or sizing can be employed.

In some embodiments, the present invention treats Neuropathic Pain, said method comprising: (i) identifying a patient with Neuropathic Pain; and (ii) administering of the present invention to a patient; wherein said patient experiences reduced pain symptoms due to said administration, with reduced side effects, and a pleasing experience.

While the making and using of various embodiments of the present invention are discussed in detail, it should be appreciated that the present invention provides many applicable inventive concepts, procedures and methods that can be embodied in a wide variety of specific contexts. The specific embodiments discussed herein are merely illustrative of specific ways to make and use the invention and do not limit the scope of the invention. The active ingredients are not limited to one scope of the invention; the active ingredient(s) are the botanical mixture, delivery system and can vary in amount, volume, and strength.

To facilitate the understanding of this invention, a number of terms are defined below. Terms defined herein have meanings as commonly understood by a person of ordinary skill in the areas relevant to the present invention. Terms such as “a”, “an”, and “the” are not intended to refer to only a singular entity but include the general class of which a specific example may be used for illustration. The terminology herein is used to describe specific embodiments of the invention, but their usage does not delimit the invention, except as outlined in the claims.

The publications and other materials used herein to illuminate the background of the invention and in particular cases, to provide additional details respecting the practice, are incorporated by reference.

It should be understood the foregoing detailed description is for purposes of illustration rather than limitation of the scope of protection accorded this invention, and therefore the description should be considered illustrative, not exhaustive. The scope of protection is to be measured as broadly as the invention permits. While the invention has been described in connection with preferred embodiments, it will be understood that there is no intention to limit the invention to those embodiments. On the contrary, it will be appreciated that those skilled in the art, upon attaining an understanding of the invention, may readily conceive of alterations to, modifications of, and equivalents to the preferred embodiments without departing from the principles of the invention, and it is intended to cover all these alternatives, modifications and equivalents. Accordingly, the scope of the present invention should be assessed as that of the appended claims and any equivalents falling within the true spirit and scope of the invention.

The inventor claims rights to obvious embodiments of this invention, including using delivery systems, compositions, combinations and solvent extraction methods as outlined in this author's patent, which is a matter of record at the United States Patent and Trademark Office as well as methods and compositions known to the art of pharmaceutical science and drug discovery.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any and all variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

No limitations with respect to the specific embodiments and examples disclosed herein are intended or should be inferred, as the examples and embodiments are representative only. While examples and preferred embodiments of the present invention have been shown and described, it will be apparent to those skilled in the art, or ascertainable using no more than routine experimentation, that many changes and modifications may be made without departing from the invention in its broader aspects. The appended claims are therefore intended to cover all such changes, modifications, and equivalents as fall within the true spirit and scope of the invention.

1. A composition having nutritional or mood-enhancing properties when administered to a person in need thereof, said composition comprising a mushroom or extract thereof selected from the group consisting of Basidiomycota mushroom, Ascomycetes mushroom, and Sordariomycetes mushroom.

2. The composition of claim 1, wherein the mushroom is Basidiomycota.

3. The composition of claim 1, wherein the mushroom is an Ascomycota.

4. The composition of claim 1, wherein the mushroom is a Sordariomycetes mushroom.

5. The composition of claim 1, further comprising cactus or an extract thereof.

6. The composition of claim 5, wherein the cactus is Opuntia ficus-indica.

7. The composition of claim 1, further comprising a cannabinoid.

8. The composition of claim 1, wherein the cannabinoid is CBD.

9. The composition of claim 5 wherein the species of mushroom or cactus is selected from the group consisting of Hericium erinaceus, Tremella fuciformis, Agaricus blazei, Cordyceps sinensis, Cordyceps militaris, Flammulina velutipes, Ganoderma lucidum, Grifola frondosa, Letinula edodes, Phellenis linteus, Polyporus umbellatus, Inonotus obliquus, Pleurotus ostreatus, P. eryngii, P. citrinopileatus, P. djamor, Trametes versicolor, Volvariella volvacea, inonotus, Hypsizygus marmoreus, Pholiota nameko, Tuber melanosporum, Moschella hortensis, Bacopa Monnieri, Sarcodon spp., Antrodia camphorata, Pleurotus giganteus, Lignosus rhinocerotis, obliquus, Trametes versicolor, Poria cocos, and combinations thereof.

10. The composition of claim 1, wherein the composition comprises:
0.15-50 wt % active ingredient compound and botanical mixture;
one or more, thickening or binding agent;
a stabilizer;
at least one fruit/vegetable juice and/or extract; and
a sweetener, enhancement, vitamins, mineral, amino acids, fruit, vegetable, herbal, essential oils/extracts, or a derivative thereof
wherein the composition is prepared as a solid dosage form selected from a troche or lozenge, confection, confection like, gummy, tablet, or chewable tablet having 0.5-6 grams in weight.

11. The composition of claim 1, wherein the composition comprises:
(I) 0.15-50 wt % active ingredient compound and botanical mixture
(II) one or more, thickening binding agents; stabilizers
(III) at least one fruit/vegetable juice and/or extract;
(IV) a sweetener, and
(V) vitamins, mineral, amino acids, fruit vegetable herbal, essential oils/extracts, or a derivative thereof
wherein the composition is prepared as a liquid beverage in a serving container having a volume of 0.2 ml to 480 ml.

12. A method for treating a person in need of antioxidant, antimicrobial, anti-inflammatory neuroregenerative, anticancer, anti-epileptic, immunomodulating, antipsychotic, psychiatric, memory enhancement, cognitive function enhancement, performance enhancement, libido enhancement, or hydration treatment, said method comprising:
providing a composition comprising a mushroom or extract thereof, the mushroom or extract being selected from the group consisting of Basidiomycota mushroom, Ascomycetes mushroom, and Sordariomycetes mushroom; and
administering an effective amount of the composition to the patient.

13. A method for myceliation of an agricultural substrate for producing a composition comprising a mushroom or extract thereof from Basidiomycota mushroom, Ascomycetes mushroom, and Sordariomycetes mushroom, said composition having nutritional or mood-enhancing properties when administered to a person in need thereof, said method comprising the steps of:
a. inoculating a fortified, agricultural grain substrate, in water or an aqueous medium with an aliquot of fungal culture selected from a group consisting of Basidiomycota and Ascomycota derived from spore, culture or liquid state fermentation to provide an optimized growth medium for fungal growth;
b. allowing mycelial growth under optimum growth conditions to obtain a myceliated substrate;
c. harvesting the myceliated substrate along with metabolized contents of the agricultural grain substrate after the mycelia growth reaches a desired stage; and
d. processing a live or dried myceliated substrate with one or more amendments to the medium which can be distilled water, spring water, filtered water, fruit or vegetable juice, broth, slurry, or gel, or infusion thereof, mixed or agitated with agar, gelatin, pectin, vegetable, grain, vegetable starch, nutritional yeast, malt extracts, dextrose, citric acid, ascorbic acid, honey, terpenes, cane sugar, essential oils, and combinations thereof.

14. The method of claim 18, wherein the process includes pre-processing steps of:
i. heat treatment comprising pasteurization, heat sterilization and filtration;
ii. optionally, a partial coating of a surface of substrate with at least one myceliation enhancing compound;
iii. a substrate known to exhibit neuroregenerative and neuroprotective properties.

15. A beverage product providing beneficial properties to a person ingesting the beverage, wherein the beverage is prepared by the steps of:
a) admix Saw Palmetto honey, wood, seeds, bark, botanicals in a sanitized mason jar and place the mason jar with admixture into a 110-degree water bath for 12 days to achieve a decarboxylation process required to activate THC;
b) add to the admixture from step a), 1 part of a floral tea damiana to 3 parts water and place in a 110-degree water bath for an additional 12 hours;
c) strain to remove the solid components.
